SEQUENCE LISTING (70 sequences)

SEQ ID NO: 1 - DNA, 21 bp, Homo Sapien
tggcgcctcatcccttggatg

SEQ ID NO: 2 - DNA, 21 bp, Homo Sapien
cagtcacccacttctgcttcg

SEQ ID NO: 3 - DNA, 22 bp, Homo Sapien
tcaacacgacaccggacaaacc

SEQ ID NO: 4 - DNA, 21 bp, Homo Sapien
ggtacttccccaacacgctcg

SEQ ID NO: 5 - DNA, 22 bp, Homo Sapien
aaggtcatcatctgccatcgag

SEQ ID NO: 6 - DNA, 21 bp, Homo Sapien
agccggcctgtgagagctttc

SEQ ID NO: 7 - DNA, 21 bp, Homo Sapien
gtcgaatacatccgcgcgctg

SEQ ID NO: 8 - DNA, 21 bp, Homo Sapien
agaaccagttggtaaagtcca

SEQ ID NO: 9 - DNA, 21 bp, Homo Sapien
gaggacatggtgctggaggag

SEQ ID NO: 10 - DNA, 21 bp, Homo Sapien
cgttgactacattcctgaagg

SEQ ID NO: 11 - DNA, 22 bp, Homo Sapien
ttatggacccaaccacgagcac

SEQ ID NO: 12 - DNA, 21 bp, Homo Sapien
gaagcgaccttctgatgtccc

SEQ ID NO: 13 - DNA, 22 bp, Homo Sapien
gcagccagtatctggatgagac

SEQ ID NO: 14 - DNA, 20 bp, Homo Sapien
ctccagcaggaggccgtggt

SEQ ID NO: 15 - DNA, 21 bp, Homo Sapien
ctggttccctgagggtttcaa

SEQ ID NO: 16 - DNA, 21 bp, Homo Sapien
ggaacttcttggtctccaggt

SEQ ID NO: 17 - DNA, 20 bp, Homo Sapien
caacatgggccgctgtcctc

SEQ ID NO: 18 - DNA, 22 bp, Homo Sapien
cacatctgcttggcagttgatc

SEQ ID NO: 19 - DNA, 21 bp, Homo Sapien
gcagctgtgaacaacgtggag

SEQ ID NO: 20 - DNA, 21 bp, Homo Sapien
aaccgcacaatgtcctggtgc

SEQ ID NO: 21 - DNA, 22 bp, Homo Sapien
aagccacgtgtaataacggtgg

SEQ ID NO: 22 - DNA, 21 bp, Homo Sapien
ctccatccacacaggttccgc

SEQ ID NO: 23 - DNA, 20 bp, Homo Sapien
ctgcctttcccatggatgaa

SEQ ID NO: 24 - DNA, 21 bp, Homo Sapien
cgcttcttgtcctcctccttt

SEQ ID NO: 25 - DNA, 19 bp, Homo Sapien
agacgacgcgaagctcacc

SEQ ID NO: 26 - DNA, 19 bp, Homo Sapien
aagccagactgcctgggct

SEQ ID NO: 27 - DNA, 20 bp, Homo Sapien
atcccaacccaccaccaacc

SEQ ID NO: 28 - DNA, 20 bp, Homo Sapien
cagcggtgcctgagaagatt

SEQ ID NO: 29 - DNA, 21 bp, Homo Sapien
aggaggaccagggactaccgt

SEQ ID NO: 30 - DNA, 21 bp, Homo Sapien
tttaggtggggtgtcactcag

SEQ ID NO: 31 - DNA, 22 bp, Homo Sapien
caaaagtccaagtgctggacaa

SEQ ID NO: 32 - DNA, 20 bp, Homo Sapien
cccatctgttgcttttcgct

SEQ ID NO: 33 - DNA, 21 bp, Homo Sapien
tgcagcacatgcagtacaacg

SEQ ID NO: 34 - DNA, 22 bp, Homo Sapien
tcccaaggttcagaaggagagg

SEQ ID NO: 35 - DNA, 20 bp, Homo Sapien
tcttctggcccggggtgatg

SEQ ID NO: 36 - DNA, 20 bp, Homo Sapien
agccgcgtgcttcttcctcc

SEQ ID NO: 37 - DNA, 20 bp, Homo Sapien
gccacatgcggcagctacag

SEQ ID NO: 38 - DNA, 20 bp, Homo Sapien
acacggccacggtgacaaag

SEQ ID NO: 39 - DNA, 19 bp, Homo Sapien
ccgctgtcctggctcttct

SEQ ID NO: 40 - DNA, 20 bp, Homo Sapien
ccgctgtgtttcttctgctg

SEQ ID NO: 41 - DNA, 20 bp, Homo Sapien
gcagcctttgtgaaccaaca

SEQ ID NO: 42 - DNA, 20 bp, Homo Sapien
tctgcggtcatcaaatgagg

SEQ ID NO: 43 - DNA, 22 bp, Homo Sapien
cccaagattttgtgcagtggtt

SEQ ID NO: 44 - DNA, 21 bp, Homo Sapien
gcggccaagttcttcaacaat

SEQ ID NO: 45 - DNA, 20 bp, Homo Sapien
ctggatgacagagccaggat

SEQ ID NO: 46 - DNA, 22 bp, Homo Sapien
tcaccattgccaccacatccat

SEQ ID NO: 47 - DNA, 21 bp, Homo Sapien
caagataccaggatgacggca

SEQ ID NO: 48 - DNA, 21 bp, Homo Sapien
gcctcaatagcatccgtcaca

SEQ ID NO: 49 - DNA, 21 bp, Homo Sapien
ccggatgacgccaaacttaca

SEQ ID NO: 50 - DNA, 19 bp, Homo Sapien
acaccagtgctcccgggag

SEQ ID NO: 51 - DNA, 21 bp, Homo Sapien
ggactttcttgcctgagcaga

SEQ ID NO: 52 - DNA, 21 bp, Homo Sapien
aactcggtggatggttcgtgt

SEQ ID NO: 53 - DNA, 21 bp, Homo Sapien
atgcgaccctgtgacatctca

SEQ ID NO: 54 - DNA, 21 bp, Homo Sapien
aagcccttcagcacaaagctg

SEQ ID NO: 55 - DNA, 22 bp, Homo Sapien
catgtcgctgaccaacacaaag

SEQ ID NO: 56 - DNA, 21 bp, Homo Sapien
tcgctgctgtcgtagaaagga

SEQ ID NO: 57 - DNA, 20 bp, Homo Sapien
agagctatgagctgcctgac

SEQ ID NO: 58 - DNA, 20 bp, Homo Sapien
ctgatccacatctgctggaa

SEQ ID NO: 59 - DNA, 21 bp, Homo Sapien
accacttcggccactatgtgt

SEQ ID NO: 60 - DNA, 21 bp, Homo Sapien
tcttggtcacaaaacaggcct

SEQ ID NO: 61 - DNA, 21 bp, Homo Sapien
tcaacaacctaaggacgcagg

SEQ ID NO: 62 - DNA, 22 bp, Homo Sapien
ttcacggacagaatcgaggaag

SEQ ID NO: 63 - DNA, 21 bp, Homo Sapien
cctgatgacctcgcaacagaa

SEQ ID NO: 64 - DNA, 21 bp, Homo Sapien
catgctgctcatcacatccag

SEQ ID NO: 65 - DNA, 21 bp, Homo Sapien
gaatccaacccttgtgtcaac

SEQ ID NO: 66 - DNA, 21 bp, Homo Sapien
gcaacgtcgtcaatacacgtg

SEQ ID NO: 67 - DNA, 21 bp, Homo Sapien
cgctgcattgacctggtcaat

SEQ ID NO: 68 - DNA, 20 bp, Homo Sapien
tacatgttgcacccttgcga

SEQ ID NO: 69 - DNA, 21 bp, Homo Sapien
ggcattgctagcttctcgtgt

SEQ ID NO: 70 - DNA, 22 bp, Homo Sapien
cataacggttgatgccatcacg

(19) United States
(12) Patent Application Publication
(10) Pub. No.: US 2003/0082810 A1
(43) Pub. Date: May 1, 2003

(54) Methods for generating insulin-secreting cells suitable for transplantation

(76) Inventors: Palle Serup, Copenhagen (DK); Harry Heimberg, Flobecq (BE); Gerard Gradwohl, Plobsheim (FR)

Correspondence Address: Reza Green, Esq., Novo Nordisk of North America, Inc., Suite 6400, 405 Lexington Avenue, New York, NY, 10174-6401, US

(21) Appl. No.: 10/090,011

(22) Filed: Feb. 26, 2002

Related U.S. Application Data
(60) Provisional application No. 60/271,474, filed on Feb. 26, 2001.

(51) Int. Cl. A61K 48/00; C12N 5/08; C12N 15/85

(52) U.S. Cl. 435/455; 435/366; 424/93.21

(57) ABSTRACT

The invention relates to methods for generating insulin secreting cells from precursor stem cells or from adult pancreatic exocrine cells. The methods of the invention are useful, for example, for generation of glucose sensitive insulin-secreting β-cells suitable for transplantation, as well as for in situ development of insulin-secreting cells in a patient in need thereof. Further, the method of the invention relates to methods for preventing premature differentiation of precursor stem cells into insulin-secreting β-cells. Still further, the invention relates to assay methods for identification of compounds that prevent or activate β-cell differentiation.

### CROSS-REFERENCE TO RELATED APPLICATIONS

This application claims the benefit under 35 U.S.C. 119 of U.S. provisional application No. 60/271,474, filed Feb. 26, 2001, the contents of which are hereby incorporated in their entirety.

### FIELD OF THE INVENTION

This invention relates generally to methods of generating pancreatic islet cells, particularly insulin-secreting β-cells. More specifically, this invention relates to the use of the transcription factors ngn3 and/or NeuroD/β2 to stimulate the differentiation of precursor stem cells to α- or β-cells or the transdifferentiation of adult pancreatic exocrine cells to β-cells. The methods of the invention are useful, for example, for in vitro generation of glucose sensitive insulin-secreting β-cells suitable for transplantation, as well as for in situ development of insulin-secreting cells in a patient in need thereof. Further, the method of the invention relates to methods for preventing premature differentiation of precursor stem cells into insulin-secreting β-cells. Still further, the invention relates to assay methods for identification of compounds that prevent or activate β-cell differentiation.

### BACKGROUND OF THE INVENTION

Primordial cells that retain some degree of self-renewal and potential for differentiation are termed “stem cells.” Following blastocyst formation, “totipotent” stem cells are generated within the inner cell mass of the fertilized egg. With the onset of gastrulation, these embryonic stem cells are thought to segregate into three major lineages on the basis of their incorporation within three primary germ layers (ectoderm, mesoderm, and endoderm). It appears that their capacity to generate all cell types becomes narrowed as they mature into tissue- and organ-specific or committed cells. It was generally held in the past that stem cells assume responsibility for the subsequent growth of tissues and organs during development, and for tissue homeostasis and repair throughout life, but that the generation of cell types is limited to that of the somatic tissue or organ in which the stem cell resides. However, recent work has raised questions concerning this assumption.

Bonner-Weir et al. (2000) Proc. Natl. Acad. Sci. USA 97:7999-8004 reported expansion of human duct tissue and differentiation into islet endocrine cells in vitro under culture conditions including an overlay with Matrigel in the presence of keratinocyte growth factor (KGF). Gradwhol et al. (2000) Proc. Natl. Acad. Sci. USA 97:1607-1611 have shown that mice homozygous for neurogenin3 (ngn3) null mutation do not develop islets of Langerhans and die shortly after birth.

Peck et al. (U.S. Pat. No. 6,001,647) report in vitro growth of islets-like structures from progenitor cells.

Kagan et al. (WO 00/47720) describe methods for isolating and differentiating pancreatic progenitor cells.

### SUMMARY OF THE INVENTION

The instant invention is based in part on the identification of neurogenin3 (ngn3) as a protein capable of activating a set of genes that drives precursor cells into a β-cell differentiation pathway or the transdifferentiation of adult pancreatic exocrine cells into insulin-secreting β-cells. It is believed that Ngn3 exerts its effects by binding to a specific regulatory element(s) (the E box sequences in the NeuroD/β2 promoter) which activates specific genes required for generation of insulin-secreting β-cells.

Accordingly, in one aspect the invention features a method of generating glucose-responsive insulin-secreting β-cell-like cells (“insulin-secreting β-cells”) from progenitor stem cells. In an alternative embodiment, the invention relates to a method of generating glucose-responsive insulin-secreting β-cells from adult pancreatic exocrine cells, where adult pancreatic cells include, but are not limited to, acinar cells and duct cells, preferably adult human duct cells. In both methods, the glucose-responsive insulin-secreting β-cell-like cells may be generated by activation of the transcription factor neurogenin 3 (ngn3). As it is believed that ngn3 is transiently expressed during normal β cell maturation, in one embodiment of this aspect of the invention, insulin-secreting β-cells are preferentially generated by transient activation of endogeneous or heterologous ngn3 gene expression. Ngn3 gene expression may be induced by activation of the endogenous gene through administration of a compound capable of activating endogenous ngn3. In another embodiment, ngn3 gene expression is achieved by providing a heterologous ngn3 gene.

In another aspect of the invention, the insulin-secreting β-cells may be generated by activation of the transcription factor NeuroD/β2. In this embodiment, insulin-secreting β-cells may be generated by activation of NeuroD/β2 gene expression via administration of a compound capable of activating endogenous NeuroD/β2 or by the provision of a heterologous NeuroD/β2 gene. It is to be understood that the above methods for generating insulin-secreting β-cells via the activation of ngn3 and/or NeuroD/β2 gene expression may be carried out in vitro or in vivo.

When the above methods are utilized for the in vitro generation of pancreatic islet cells, cultured progenitor stem cells or adult pancreatic exocrine cells are contacted with a compound which activates pancreatic ngn3 and/or NeuroD/β2 gene expression, preferably transient gene expression. Transient expression of neurogenin 3 gene expression is achieved in one embodiment, by a compound with capacity to transiently induce, e.g., turn on, ngn3 and/or NeuroD/β2 gene expression or under conditions in which ngn3 gene activation is subsequently inhibited, e.g., turned off. In another embodiment, transient ngn3 gene expression is achieved by introduction of a heterologous ngn3 gene into the cells via, for example, the Adeno-X Tet-Off Gene Expression System® (Clontech) or with the recombinant adenoviruses described in Example 5B. Alternatively, NeuroD/β2 gene expression is achieved by a compound with the capacity to activate NeuroD/β2 gene expression or by the introduction of a heterologous NeuroD/β2 gene into the cells via, for example, the Adeno-X Tet-Off Gene Expression System® (Clontech) or with the recombinant adenoviruses described in Example 5B.

The invention therefore relates to pancreatic islet cells, and preferably insulin-secreting β-cells, generated in vitro and their use in transplantation to a patient in need of such cells. The invention thus features a method of providing pancreatic islet cells useful for transplantation. In one embodiment, the pancreatic islet cells are generated in vitro from progenitor stem cells obtained from the patient into which the islet cells will be transplanted. In an alternative and preferred embodiment, the pancreatic islet cells are generated in vitro from adult pancreatic exocrine cells obtained from the patient into which the islet cells will be transplanted. The cells to be transplanted may be genetically modified, oncogene-suppressed or removed prior to transplantation into a patient in need thereof.

The invention further features a therapeutic method for in vivo generation of pancreatic islet cells in a patient in need thereof. In one embodiment, the invention comprises administering to the patient a compound which activates pancreatic ngn3 and/or NeuroD/β2 gene expression.

In an alternative embodiment, the invention further features a gene therapeutic method for generating in vivo pancreatic α- and/or β-cells in a patient in need thereof comprising administering the ngn3 and/or NeuroD/β2 gene to the patient.

The invention also features methods for directing overgeneration of one pancreatic cell type relative to another. In this embodiment, culture conditions and protocols are selected to direct generation of one pancreatic cell type relative to another, for example, to generate a larger proportion of insulin-secreting β-cells relative to non-insulin secreting α-cells by inducing transient expression of ngn3. In another embodiment, culture conditions are selected to direct the generation of a larger proportion of non-insulin secreting cells (e.g., α-cells) relative to other cell types, by inducing non-transient ngn3 expression, and/or by varying culture medium components. In another aspect, the invention features an assay system for identifying compounds that activate ngn3 and/or NeuroD/β2 gene expression. In one embodiment of the method of identifying compounds that activate ngn3 and/or NeuroD/β2 gene expression, cells such as precursor stem cells or adult pancreatic exocrine cells are incubated with and without the test compound, and the ability of the test compounds to activate ngn3 and/or NeuroD/β2 gene expression is measured by the ability of the test compound to generate insulin-secreting β-cells from the treated cells. Compounds identified by these methods may be useful in generating insulin-secreting β-cells from precursor stem cells or adult pancreatic exocrine cells.

In a related aspect, the invention features an assay system for identifying compounds with specifically inhibit ngn3 and/or NeuroD/β2 expression. Such compounds are useful, for example, in in vitro culture systems for inhibiting premature differentiation of precursor stem cells into pancreatic cells.

Other aspects of the invention will become apparent from the following detailed description and the claims.

### DESCRIPTION OF THE FIGURES

FIGS. 1A and 1B show the endogenous expression of key developmental transcription factors in adult human pancreatic duct cells.

FIG. 1A shows the results of RT-PCR analysis of RNA extracted from adult human duct cells and adult human islet cells and from cells where no reverse transcription was carried out (indicated by the “-”symbol). The beta-actin RNA levels were measured as an internal standard for comparison of the differences in the levels of expression of the different transcription factors between the adult human duct cells and adult human islet cells.

FIG. 1B (top panel) shows the results of immunoblots of protein extracts of MIN6 cells (a mouse insulinoma line that serves as a positive control for Nkx6.1 expression) and adult human duct and islet cells using anti-Nkx6.1 antibodies. FIG. 1B (lower panel) shows the results of immunohistochemical analysis of sections of the adult human pancreas with anti-Nkx6.1 antibodies (“Nkx6.1”) in combination with either anti-insulin (“Ins”) or anti-cytokeratin (“CA 19.9”) antibodies.

FIG. 2 shows the expression of ngn3, NeuroD/β2 and green fluorescent protein (“GFP”) in noninfected adult human duct cells (“noAd”) and in adult human duct cells infected with the recombinant adenoviral vectors AdGFP, Adngn3 and AdNeuroD/β2. The “Adngn3” referred to in the upper panel of FIG. 2 and the “Adngn3/GFP” referred to in the lower right panel of FIG. 2 are the same vector, ie a vector that coexpresses ngn3 and GFP).

The upper panel of FIG. 2 shows the results of immunoblots of protein extracts (2 ug of total protein) from noninfected adult human duct cells (“noAd”) and adult human duct cells infected with the recombinant adenoviral vectors AdGFP, Adngn3 and AdNeuroD/β2 using anti-ngn3, anti-NeuroD/β2 and anti-GFP antibodies.

The lower panel of FIG. 2 shows monolayers from infected (AdGFP or Adngn3/GFP) and non-infected (noAd) adult human duct cells (bar=10 μm) that were immunostained with anti-ngn3, anti-GFP and anti-DAPI antibodies where the anti-DAPI antibodies stain the nuclei of all cells.

FIGS. 3A-3C show the effects of adenovirus-mediated ectopic expression of ngn3 or NeuroD/β2 on key transcription factors and signal transduction proteins in adult human duct cells.

FIG. 3A shows the results of RT-PCR analysis of RNA encoding key developmental transcription factors (NeuroD/B2, Pax4, Nkx2.2, Nkx6.1, mo ngn3 and rt NeuroD/β2) from islets and from control (“no Ad”) and virus-infected (“AdGFP”, “Adngn3” or “Ad NeuroD/β2”) duct cells. “mo ngn3” indicates mouse ngn3 and “rt NeuroD/β2” indicates rat NeuroD/β2 and B-actin RNA levels were measured as an internal standard for comparison of the differences in the levels of expression of the different transcription factors between the different cells The “-” symbol indicates cells in which no reverse transcription was carried out. FIG. 3B shows the results of an analysis of the effects of ngn3 (b.,d.,f.,h.,j.) compared to AdGFP-infected control cells (a.,c.,e.,g.,i.) at the cellular level by in situ hybridisation of NeuroD/β2 (a.,b.) and Pax4 (c.,d.) mRNA and immunofluorescence for Nkx2.2 (e.-h.). GFP was detected by fluorescence. Arrowheads in panel f. point to cells expressing either Nkx2.2 or ngn3 (intense red resp. green nuclear staining) and arrow points to a cell expressing Nkx2.2 and still containing GFP (weak green fluorescence in cytoplasm combined with weak red staining in the nucleus) without high level expression of ngn3. Panels g. and h. represent a combination of phase-contrast and fluorescence microscopy Nkx2.2-immunostaining), emphasizing the massive effect of ngn3. (bar=10 μm) FIG. 3C shows the results of RT-PCR analysis of RNA encoding mouse ngn3 (“mo ngn3”), B-actin and Delta (Dll1, Dll3 and Dll4) and Notch (Notch 1, 2 and 3 and Jagged 1 and Jagged 2) isoforms from islets and from control (“no Ad”) and virus-infected (“AdGFP”, “Adngn3”) duct cells. The “-” symbol indicates cells in which no reverse transcription was carried out.

FIGS. 4A and 4B show the effect of adenovirus-mediated expression of ngn3 and NeuroD/β2 in the rat neuroendocrine cell line PC12.

FIG. 4A shows the results of RT-PCR analysis of RNA encoding B-actin, key developmental transcription factors and insulin from rat beta cells (“rat B”) and from control (“no Ad”) and virus-infected (“AdGFP”, “Adngn3”, “AdNeuroD/β2”) PC12 cells The “-” symbol indicates cells in which no reverse transcription was carried out. FIG. 4B shows immunostaining of insulin in PC12 cells that were infected at low MOI with Adngn3. Arrow points to insulin-positive PC12 cell that still contains lots of active GFP, arrowheads point to PC12 cells that express insulin but contain no active GFP or ngn3 anymore. (bar=10 μm).

FIGS. 5A and 5B show the effect of adenovirus-mediated ectopic expression of ngn3 or NeuroD/β2 on markers of the differentiated endocrine phenotype in adult human duct cells.

FIG. 5A shows the results of RT-PCR analysis of RNA encoding B-actin or human endocrine marker proteins [synaptophysin (“Syn”), chromogranin A (“CgA”), prohormone convertase 1/3 (“PC1/3”), glucokinase (“Gck”) and insulin in islets and in control (“no Ad”) and virus-infected (“AdGFP”, “Adngn3”, “AdNeuroD/β2”) adult human duct cells. The “-” symbol indicates cells in which no reverse transcription was carried out.

FIG. 5B shows immunostaining of control-(AdGFP-) (panels a. and e.) and Adngn3-(panels b.-d., f.-j.) infected duct-cells. Nuclei stained blue by DAPI (panels a.,b.,d.,h.,j.). All immunoreactions were labeled with a fluorescent secondary antibody, except for e. and f. that were immunochemically stained (ABC-peroxidase). Panels c. and g. represent a combination of phase-contrast and fluorescence microscopy. Non- and control-infected duct cells contained low number of endocrine cells (<1% insulin-positivity and <2% synaptophysin-positivity). Endocrine marker proteins were chromogranin A (CgA), synaptophysin (Syn), prohornone convertase 1/3 (PC1/3) and insulin (Ins); duct cell marker was cytokeratin 19 (CK19). Non-infected and GFP control-infected duct cells contained none of the endocrine proteins under study as determined by co-staining for the duct cell markers CK19 or CA19.9 (bar=10 μm) Arrowheads: single-positive cells (panels b. and c.: either ngn3 or CgA; panel h.: insulin); arrows: cells co-expressing CgA and ngn3 (panel b.) or insulin and ngn3 (panel h.).

### DETAILED DESCRIPTION OF THE INVENTION

Before the methods of the invention are described, it is to be understood that this invention is not limited to the particular methods described. The terminology used herein is for the purpose of describing particular embodiments only, and is not intended to be limiting since the scope of the present invention will be limited only by the appended claims.

As used in this specification and the appended claims, the singular forms “a”, “an”, and “the” include plural references unless the context clearly dictates otherwise. Thus, for example, reference to “the formulation” or “the method” includes one or more formulations, methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned herein are incorporated herein by reference for the purpose of disclosing and describing the material for which the reference was cited in connection with.

Definitions

The term “an insulin-secreting cell” means a cell which secretes insulin, such as, for example, a pancreatic β-cell, but also includes a cell manipulated to express an homologous or heterologous insulin gene where a “cell manipulated to express an homologous or heterologous insulin gene” means a cell that is infected with a heterologous ngn3 and/or NeuroD/β2 gene or treated with a compound capable of activating endogenous ngn3 and/or NeuroD/β2 gene expression in order to trigger expression of an homologous or heterologous insulin gene that is present in the cell. A “glucose-responsive” or “glucose-sensitive” insulin-secreting cell refers to a cell that is capable of modulating insulin secretion in response to environmental glucose concentrations, e.g., insulin secretion is increased in the presence of high (for example, 10 mM) blood glucose levels, and decreased in the presence of low blood glucose levels (for example, 1-3 mM blood glucose).

The term “culture medium” generally refers to any substance or preparation used for the cultivation of living cells.

The term “differentiation” and the like, is meant to refer to the process in which progenitor cells that lose pluripotency and become recognizable as lineage committed cells, displaying markers associated with a specific cell type, e.g., pancreatic cell. A more fully differentiated cell will display more specific cell characteristics and functions than a less differentiated cell, e.g., an insulin-secreting cell is more differentiated than a stem cell.

A progenitor cell is an undifferentiated cell which is capable of proliferating and giving rise to more progenitor cells having the ability to generate a large number of mother cells that can in turn give rise to differentiated, or differentiable daughter cells. As used herein, the term “progenitor cell” is also intended to encompass a cell referred to as a “stem cell” or a “progenitor stem cell.” Progenitor cells refer to a generalized mother cell whose descendants specialize by differentiation, e.g., by acquiring completely individual characteristics, as occurs in progressive diversification of embryonic cells and tissues. Progenitor or stem cells refers to cells arising in tissue of a pancreatic intralobular duct and giving rise to such differentiated progeny as, for example, β-cells.

The term “activation of gene expression” and the like means activation of ngn3 and/or NeuroD/β2 gene expression resulting in production of the ngn3 and/or NeuroD/β2 protein. The ngn3 protein and/or NeuroD/β2 protein bind to regulatory elements of specific genes, thus in turn activating gene expression which results in directed differentiation of precursor stem cells into insulin-secreting β-cells or in the transdifferentiation of adult pancreatic exocrine cells into insulin-secreting β-cells. In one embodiment, expression of ngn3 and/or NeuroD/β2 is assured by inserting a regulatory element(s) into a virus vector capable of driving expression of the ngn3 and/or NeuroD/β2 gene in cells infected by the virus.

The term “transient” expression of, for example, ngn3, means ngn3 gene expression is activated for a short period of time. In one embodiment, transient gene expression is achieved with the use of Adeno-X Tet-Off Gene Expression System® (Clontech).

### GENERAL ASPECTS OF THE INVENTION

Directed Differentiation of Precursor Stem Cells into Insulin-Secreting Cells

This invention is based, in part, on the discovery that expression of the transcription factor neurogenin 3 (ngn3) is required for directed differentiation of precursor stem cells into insulin-secreting cells. Accordingly, the invention features a method of generating insulin-secreting cells from precursor stem cells by activation of ngn3 gene expression. In a specific embodiment, ngn3 gene expression is transiently activated in cell capable of developing into insulin-secreting cells, such that insulin-secreting β-like cells are generated in greater proportion than in the absence of transient ngn3 expression.

Ngn3, a member of the basic helix-loop-helix (bHLH) family of transcription factors, acts upstream of NeuroD in a bHLH cascade. Detailed analysis of ngn3/PAX6 and NeuroD/PAX6 co-expression shows that the two bHLH factors are expressed in a largely non-overlapping set of cells, but such analysis also suggests that the NeuroD⁺ cells arise from cells expressing ngn3 transiently. NeuroD⁺ cells do not express Ki-67, a marker of proliferating cells, which shows that these cells are postmitotic. In contrast, Ki-67 is readily detected in ngn3⁺ cells (Jensen et al. (2000) Diabetes 49:163-176). Endocrine cells are not formed in ngn3 mutant mice (Gradwohl et al. (2000) PNAS USA 97:1607-11). Thus, ngn3⁺ cells fulfill the criteria for an endocrine precursor cell, and it is believed that α-cell and β-cells develop independently from PDX1⁺/ngn3⁺ epithelial cells, rather than from GLU⁺/INS⁺ intermediate stages.

The work described below shows a temporal sequence of gene activation and inactivation for developing α- and β-cells from precursor stem cells, which begins with the activation of NeuroD expression.

It is also believed that expression of the transcription factor NeuroD/β2 will direct the differentiation of precursor stem cells into insulin-secreting cells. Accordingly, the invention features a method of generating insulin-secreting cells from precursor stem cells by activation of NeuroD/β2 gene expression. In a specific embodiment, NeuroD/β2 gene expression is activated in cell capable of developing into insulin-secreting cells, such that insulin-secreting β-like cells are generated in greater proportion than in the absence of transient NeuroD/β2 expression.

Accordingly, the instant invention in part encompasses culture conditions which mimic in vivo pancreatic cell development from precursor stem cells.

This invention further encompasses the prevention of premature β-cell differentiation from precursor stem cells with culture conditions which inhibit a ngn-NeuroD cascade through activation of Notch1 expression.

Directed Transdifferentiation of Adult Pancreatic Duct Cells into Insulin Secreting Cells

While much of the current emphasis is placed on the differentiation of embryonic stem cells to insulin-producing cells {Lumelskyet al. Science 292: 1389-1394 (2001)} {Assadyet al. Diabetes 50: 1691-1697 (2001)}, transdifferentiation of adult duct cells can provide an abundant β-cell source for transplantation, avoiding the ethically questionable use of embryonic stem cells and getting around immune incompatibility when isografted.

This invention is therefore also based in part on the discovery that primary duct cells, isolated from adult human pancreas, can be converted into insulin-expressing cells by adenovirus-mediated expression of the bHLH factor neurogenin-3 (ngn3), a critical factor for embryonic endocrinogenesis in the pancreas. A similar induction was observed in the neuroendocrine cell line PC12 but not in HeLa cells. Infection of the adult duct cells with ngn3 induced the expression of NeuroD/β2, Pax4 and Nkx2.2, all essential for β-cell differentiation but silent in control duct cells. Furthermore, the expression of the Notch ligand Dll1 and Dll4 was induced in duct cells by ngn3, suggesting that the Delta-Notch pathway of lateral inhibition, which controls embryonic development of the endocrine pancreas, applies as well to the post-natal endocrine differentiation program.

NeuroD/β2, an immediate downstream target of ngn3, by itself also activated Pax4 and Nkx2.2 and converted primary duct cells, isolated from adult human pancreas, into insulin-expressing cells. The expression of several neuroendocrine-specific markers including synaptophysin and chromogranin A, and the β-cell markers PC1/3, glucokinase and insulin was activated both by ngn3 and NeuroD/β2. Taken together, this data i) suggest that the Delta-Notch program for embryonic development of the endocrine pancreas is implicated in post-natal endocrine differentiation, (ii) demonstrate the induction of the multistep process of endocrinogenesis by expression of a single key transcription factor in duct cells of the adult human pancreas, and (iii) extend previous studies of embryonic pancreas formation in transgenic mice yet confine the endocrinogenic effect of ngn3 in adult human duct cells to differentiation towards β-cells.

The present invention therefore relates to methods of generating insulin-secreting β-cells (preferably generating glucose-responsive insulin-secreting β-cells) from adult pancreatic exocrine cells, preferably from adult human duct cells. In these methods, the insulin-secreting β-cells may be generated by activation of the transcription factor neurogenin 3 (ngn3) and/or NeuroD/β2. In these methods, insulin-secreting β-cells may be generated by transient activation of ngn3 and/or NeuroD/β2 gene expression via administration of a compound capable of activating endogenous ngn3 and/or NeuroD/β2 or by the provision of a heterologous ngn3 and/or NeuroD/β2 gene.

Activation of Ngn3 and/or NeuroD/β2 through the use of Transient Gene Expression

In one embodiment of the invention, ngn3 and/or NeuroD/β2 are transiently expressed in cells through recombinant gene expression. The complete coding sequence of the mouse and human ngn3 genes is found at GenBank accession numbers U76208 and XM 005744 respectively while the complete coding sequence of the human and rat NeuroD/β2 genes is found at GenBank accession numbers U50822 and AF107728 respectively

In one embodiment, transient gene expression of ngn3 is achieved by co-infecting cells with two different recombinant adenovira, Virus-1 and Virus-2. Virus-1 encodes a tetracyclin regulated transactivator (tTA) under control of a constitutive promoter. Expression of tTA in the cells allows expression of ngn3 from Virus-2, encoding ngn3 under control of a tetracyclin response element containing promoter TRE-pCMVmin. In the presence of tetracyclin or tetracyclin analogs, tTA is rendered inactive, thus after a designated period of time, tetracyclin (or a tetracyclin analog) is added to the cells, resulting in repression of ngn3 expression.

In another embodiment, transient ngn3 expression is achieved by replacing Virus-1 with a virus that encodes the reverse tetracyclin regulated transactivator (rtTA). rtTA is only active in the presence of tetracyclin. This virus is co-infected with a recombinant virus encoding ngn3 under control of the TRE-pCMVmin promoter. Ngn3 expression is induced by addition of tetracyclin or tetracyclin analogs for a designated period of time.

In yet another embodiment, ngn3 and NeuroD/β2 are transiently expressed using the adenoviral vectors Adngn3 and Ad NeuroD/β2 described in Example 5.

Of course, one skilled in the art would readily understand that there are a variety of methods and vectors available other than those described above that are suitable for introducing a nucleic acid molecule encoding ngn3 or NeuroD/β2 into cells in vitro or in vivo.

Activation of Ngn3 and/or NeuroD/β2 through the use of Compounds which are Activators of Ngn3 and/or NeuroD/β2 Gene Expression

Compounds which are activators of ngn3 and/or NeuroD/β2 gene expression include, but are not limited to, transcription factors that bind to the promoters of the ngn3 or NeuroD/β2 genes and activate their transcription as well as compounds that may indirectly induce activation of ngn3 and/or NeuroD/β2 gene expression. An example of an activator of ngn3 includes the transcription factor hepatocyte nuclear factor 6 (HNF6) which has been shown to bind to and stimulate the ngn3 promoter. [Jacquemin et al (2000) Mol. Cell. Biol. 20: 4445-4454] and an example of an activator of NeuroD/β2 is ngn3 which has been shown to bind to the NeuroD/β2 promoter (see Example 5B).

Of course, the identification of additional compounds as activators of ngn3 and/or NeuroD/β2 gene expression may be achieved through the use of cells transfected with constructs containing the ngn3 or NeuroD/β2 promoters linked to a reporter gene such as B-galactosidase or chloramphenicol acetyltransferase. In such an assay, the ability of a test compound to activate NeuroD/β2 or ngn3 gene expression in the transfected cells is measured by the ability of the test compound to induce expression of the reporter gene. Alternatively, such compounds are identified through the use of the screening assays described below.

Culture Medium and Conditions for Generating Insulin-Secreting Cells

In one aspect, the invention features the use of defined culture conditions for generating specifically desired cells, e.g., glucose-responsive insulin-secreting cells. Prior art methods are known for isolating and propagating progenitor cells, e.g., pancreatic ductal stem cells. See, for example, WO 00/47720 (PCT/US00/03419) the specification of which is specifically herein incorporated by reference in its entirety for describing methods of isolating and propogating pancreatic progenitor cells. As described in WO 00/47720, small amounts of starting material, such as human adult pancreatic tissue can be obtained from a donor without serious injury to the donor. From the starting material, progenitor cells can be obtained and amplified under culture conditions and media which may include a cAMP elevating agent, a growth factor, and a steriod or corticosteroid. Once progenitor cells are obtained, it is generally desirable to cause proliferation of the progenitor cell population, such that the number of cells available to differentiate into a desired cell type, e.g., a glucose-responsive insulin-secreting β-like cell, are increased. Depending on the desired cell type to be generated, culture media will contain selected growth factors and the culture conditions will expose the cells to selective transcription factors. The experiments described below identify culture conditions and media components required for the generation of insulin-secreting cells from precursor stem cells. The candidate growth factors tested are selected from the following:

Fibroblast Growth Factor.

The effect of human keratinocyte growth factor (KGF/FGF-7) expression in the embryonic liver of transgenic mice was examined (Nguyen et al. (1996) Oncogene 12:2109-2119). Among the effects observed was a significant hyperplasia in pancreatic duct cells and an increased number of ductal epithelial cells staining positive for insulin expression. Systemic administration of exogenous KGF to adult rats has been observed to stimulate growth and differentiation of epithelial cells in many tissues, including the pancreas (Housley et al. (1994) J. Clin. Invest. 94:1764-1777). More recently, observations have been made that suggest that tissue-specific stem cells, even in adult tissue, may exhibit characteristics of a different environment when placed outside their normal residence. Intra-germal plasticity may result in stem cells giving rise to derivatives of the same germ layer, e.g., mesenchymal stem cells yielding cartilage, bone, and adipocytes (Pittinger (1999) Science 284:143-147) and bone marrow cells undergoing myogenic differentiation (Jackson et al. (1999) Proc. Natl. Acad. Sci. USA 96:14482-14486). The possibility of inter-germ layer transdifferentiation may be evidenced by differentiation of ectoderm-derived neural stem cells into mesoderm-derived hematopoietic cells (Bjornson et al. (1999) Science 283:534-537) or neuroectoderm-derived glial cells (Kopen et al. (1999) Proc. Natl. Acad. Sci. USA 96:10711-10716).

TGF-β Family.

The TGF-β superfamily has approximately 40 known members in mammals. These include activins, inhibins, bone morphogenetic proteins (BMP), growth- and differentiation factors (GDF), including TGF-β. These secreted molecules signal through a hetero-dimeric serine-threonine kinase receptor complex composed of type-1 and type-2 receptors (Massague et al. (2000) Embo J. 19(8):1745-54).

Notch ligands:

Notch signaling is important for proper development of pancreatic endocrine cells. Mice lacking the Notch pathway genes (Dll1, RBP-jκ, HES-1) showed accelerated differentiation of endocrine cells in the pancreas (Apelqvist et al. (1999) Nature 400:877-81). It is believed that Notch agonists and antagonists are useful to control proliferation and differentiation of endocrine precursor cells in vitro.

Epidermal Growth Factor (EGF) Family:

The ErbB receptor family: ErbB proteins belong to the receptor tyrosine kinase (RTK) superfamily. There are four members of the ErbB family: epidermal growth factor (EGF) receptor (also termed ErbB1/HER 1), ErbB2/Neu/HER2, ErbB3/HER3 and ErbB4/HER4, all of which are expressed in embryonic pancreatic epithelium (Miettinen et al. (2000) Development 127:2617-27). A family of ligands, the EGF-related peptide growth factors, bind the extracellular domain of ErbB receptors leading to the formation of both homo- and heterodimers. ErbB homo- and heterodimer combinations are useful for providing a high degree of signaling diversity.

Fibroblast Growth Factor (FGF) and FGFR family.

The mammalian FGF family consists of at least 23 structurally related polypeptides which play key roles in morphogenesis, development, angiogenesis, and wound healing (Yamashita et al. (2000) Biochem. Biophys. Res. Commun. 277:494-8). FGF-stimulated processes are mediated by four FGF receptor tyrosine kinases (FGFR), composed of an extracellular ligand binding portion consisting of three immunoglobulin (Ig)-like domains (D1, D2, and D3), a single transmembrane helix, and a cytoplasmic portion that contains protein tyrosine kinase activity. A number of the FGFs are expressed in the developing pancreas and are believed to be required for proper growth and differentiation of the embryonic epithelium.

Insulin-Like Growth Factors (IGF, IGF1-R, and IRS-2):

Islets are reduced in size in IGF I-R (+/−) mice, and develop poorly in IGF1-R (−/−) mice as a reduced number of α-cells and β-cells fail to form typical islet structures. The IGF1-R (−/−) mice die at birth, whereas IGF 1-R (+/−) mice survive with reduced β-cell mass, and never develop diabetes; however, combined with an IRS-2 disruption, IGF1-R (+/−)/IRS2 (−/−) mice die at 30 days of age with severe hyperglycemia, owing to small islets containing a reduced number of α-cells and nearly absent β-cells. IRS-2 plays a special role in carbohydrate metabolism as it mediates both peripheral insulin action and pancreatic β-cell function. It appears that the presence of a single allele of irs-2 promotes sufficient β-cell expansion and survival to compensate for severe peripheral insulin resistance (Withers et al. (1999) Nat. Genet. 23:32-40). The IGF1-R>IRS-2 signaling pathway is important in both development and growth of β-cells.

Hepatocyte Growth Factor (HGF).

HGF is expressed in the mesenchyme and its RTK-receptor c-Met in the epithelium. HGF stimulates growth of adult human duct cells in culture (Beattie et al. (1996) Diabetes 45:1223-8).

Glucagon-Like Peptide (GLP-1).

GLP-1 is known to function in glucose metabolism. It has a stimulatory effect on β-cell neogenesis and replication in rats and mice.

Nerve Growth Factor (NGF)/Neurotrophin.

The NGF/neurotrophin family and its cognate receptor family (TrkA-C) has several members expressed in the developing pancreas. NGF is expressed in the mesenchyme and TrkA in the embryonic epithelium, and in mature β-cells (Kanaka-Gantenbein et al. (1995) Endocrinology 136:761-9). Additionally, the NT-3 receptor TrkC is expressed in INS-1 cells (TAzi et al. (1996) J. Biol. Chem. 271:10154-60).

Wnt Family.

The Wnt morphogen family and its receptors, the Frizzled proteins have several members expressed during pancreas development (LeBras et al. (1998) Diabetologia 41:1474-81).

HlxB9.

HlxB9 is required at an early step for expression of all other known pancreatic markers in the dorsal anlage. Later in development HB9 becomes restricted to mature β-cells, where it is required for normal GLUT2 and Nkx6-1 expression (Harrison et al. (1999) Nat. Genet. 23:71-5).

PDX1.

Pdx1 (human IPF1) expression is initiated at the 10-12 somite stage, i.e., before hormone gene expression, and is restricted to the dorsal and ventral walls of the primitive foregut at the positions where pancreas will later form (Ahigren et al. (1996) Development 122:1409-16). The pattern of Pdx1 expression and its ability to stimulate insulin gene transcription suggests a role in regionalization, as well as maturation of the pancreatic β-cell. Homozygosity for mutations in the Pdx1 gene in mice and humans results in a complete pancreas agenesis (Stoffers et al. (1997) Nat. Genet. 15:106-10), and Pdx1 (−/−) embryos exhibit an arrest of endocrine cell differentiation. Later in development, Pdx1 becomes restricted to mature β-cells, where it is required for normal GLUT2 and Nkx6-1 expression (Ahlgren et al. (1998) Genes Dev. 12:1763-8).

Nkx2-2

Nkx2-2 belongs to the mammalian NK2 homeobox family of transcription factors and is expressed from at least the 14 somite stage in both the dorsal and ventral pancreatic epithelium (Edlund (1998) Diabetes 47:1817-23). Later in development, Nkx2-2 is expressed in all islet cells except the somatostatin-producing β-cells. It is believed that Nkx2-2 is required for the terminal differentiation of β-cells and/or the expression of the insulin gene.

Nkx6-1/6-21.

Nkx6-1/6-21 is initially expressed predominantly in the dorsal pancreatic epithelium in a subset of the PDX1⁺ cells (Øster et al. (1998) J. Histochemistry Cytochemistry 46:707-15). Later in development, Nkx6-1 becomes restricted to the differentiated β-cells.

NeuroD.

NeuroD/Beta2 is a cell-type restricted bHLH transcription factor that is expressed in all pancreatic endocrine cells. Together with its ubiquitous counterpart, the E2A gene product E12, NeuroD/Beta2 binds to the E-boxes of the insulin regulatory region. In NeuroD/Beta2-deficient mice, endocrine cells still appear, but at markedly reduced numbers

ISL1.

During pancreas ontogeny, the expression of the LIM homeodomain protein Isl1 is initiated soon after the islet cells have left the cell cycle. In the adult pancreas, Isl1 is expressed in all classes of islet cells. In mice lacking Isl1 function, the dorsal pancreatic mesenchyme does not form, and there is an associated failure of exocrine cell differentiation in the dorsal but, not the ventral, pancreas. In addition, there is a complete loss of differentiated islet cells. Isl1 is necessary for the development of the dorsal exocrine pancreas and is required for the generation of all endocrine islet cells.

Pax6.

Pax6 belongs to the Pax gene family and, along with Isl1, is expressed in all endocrine cells both during development and in the adult pancreas. In mice homozygous for deletions of the Pax6 gene, the numbers of all differentiated endocrine cell types are markedly reduced, and in addition, there is a significant decrease in hormone production (St. Onge et al. (1997) Nature 387:406-9).

Pax4.

Mice lacking a functional Pax4 gene lack differentiated insulin and somatostatin cell types. In addition, there is an associated increase in the number of glucagon-producing cells (Sosa-Pineda (1997) Nature 386:399-402). It is believed that Pax4 is responsible for an Ins⁺ cell fate choice, and that the default fate in the absence of Pax4 is a Glu⁺ cell.

HNF-6.

HNF-6 is a member of the cut-homeodomain family of transcription factors. HNF-6 expression can be detected as early as e10.5 in both pancreatic buds, but later, the expression becomes restricted to the exocrine pancreas. HNF-6 expression also remains in the pancreatic epithelium, but the endocrine cells are HNF-6 negative throughout development. HNF6 is believed to have a role as an activator of ngn3 expression, and in consequence HNF6 mutant animals have very few β-cells at birth (Jacquemin et al. (2000) Mol. Cell. Biol. 20:4445-54).

An example of one set of conditions suitable for increasing the population of adult duct cells suitable for transdifferentiation into insulin-secreting cells is provided in Example 5A.

Transplantation of Pancreatic Cells Generated in vitro

In one aspect of the invention, mature pancreatic cells generated in vitro by the methods of the invention are useful in transplantation therapies. Such transplantation therapy is believed to be useful in the treatment of diseases such as diabetes where the transplantation of mature pancreatic cells, preferably islet cells, provides a means of restoring euglycemia and insulin independence that avoids the surgical complications of pancreatic transplantation. Indeed, the implantation of islets derived in vitro from pancreatic ductal epithelial cells has been demonstrated to reverse diabetes in a mouse model system. [Ramiya et al (2000) Nat. Med. 6: 250-251).

Thus, in one embodiment, the cells generated by the methods of the invention are transplanted into a patient in need thereof, e.g., in vitro generated pancreatic islet cells are transplanted to replace destroyed or malfunctioning cells in the patient. The pancreatic islet cells are preferably of the same species as the host into which they will be transplanted. Generally, mammalian cells are used for treating mammalian subjects. Thus, in the case of a human patient, the cells are preferably human.

In one aspect of cell transplantation, transplanted cells serve to supplement the cells that are destroyed, malfunctioning, or absent in the transplant patient. In the second aspect, In the second case, the transplanted cells contain a vector with a foregin gene expressing a desired product that is missing, malfunctioning, or expressed at a low level in the transplant patient, and the transplanted cells are transfected with a vector containing DNA encoding the desired product.

In the instant method of the invention, transplanted cells are preferably generated from precursor stem cells obtained from the patient to receive the cells. Preferably, the transplanted cells secrete insulin in response to glucose level in the patient, in an amount and with the precise timing that is needed for control of glucose homeostasis.

Methods are known to the art for administering differentiated cells, particularly to a human subject in need thereof, including injection, implantation, insertion into a delivery device which facilitates introduction into the host subject.

Tranplantation of generated β-cells, glucose-responsive insulin secreting cells, and the like, may include encapsulation in a membrane to avoid immune rejection. Many methods of encapsulation are known in the art. See, for example, WO 00/47720.

### In vivo Production of Insulin Secreting Cells

As discussed above, activation of ngn3 and/or NeuroD/β2 gene expression may also be used to generate insulin secreting cells in vivo in a patient in need of such treatment where the terms “treatment”, “treating” and the like are used herein to generally mean obtaining a desired pharmacologic and/or physiologic effect. The effect may by prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, particularly a human, and includes:

(a) preventing the disease from occurring in a subject which may be predisposed to the disease but has not yet been diagnosed as having it;

(b) inhibiting the disease, i.e., arresting its development; or

(c) relieving the disease, i.e., causing regression of the disease. The invention is directed in part to treating patients with or at risk for development of a pancreatic hormone deficiency, such as diabetes, and related conditions mediated by insulin insufficiency. More specifically, “treatment ”is intended to mean providing a therapeutically detectable and beneficial effect on a patient suffering from, for example, a condition mediated by insulin-secreting β-cells and more particularly, by a condition mediated by the destruction of insulin-secreting β-cells.

When activation of ngn3 and/or NeuroD/β2 gene expression is to be achieved through the administration of a compound able to activate pancreatic ngn3 and/or NeuroD/β2 gene expression, the compound is administered to the patient in a therapeutically effective amount.

By the term “effective amount” or “therapeutically effective amount” of a compound able to activate pancreatic ngn3 gene expression is meant an amount of a compound sufficient to obtain the desired physiological effect, e.g., generation or restoration of, for example, insulin-secreting β-cells. An effective amount of such a therapeutic compound is determined by the caregiver in each case on the basis of factors normally considered by one skilled in the art to determine appropriate dosages, including the age, sex, and weight of the subject to be treated, the condition being treated, and the severity of the medical condition being treated.

The terms “synergistic”, “synergistic effect” and like are used herein to describe improved treatment effects obtained by combining one or more therapeutic agents with one or more neurogenin 3 or NeuroD/β2 gene activating compounds. Although a synergistic effect in some field is meant an effect which is more than additive (e.g., 1+1=3), in the field of medical therapy an additive (1+1=2) or less than additive (1+1=1.6) effect may be synergistic. For example, if each of two drugs were to inhibit the development of pancreatic islet cell insufficiency by 50% if given individually, it would not be expected that the two drugs would be combined to completely stop the development of pancreatic islet cell insufficiency. In many instances, due to unacceptable side effects, the two drugs cannot be administered together. In other instances, the drugs counteract each other and slow the development of pancreatic islet cell insufficiency by less than 50% when administered together. Thus, a synergistic effect is said to be obtained if the two drugs slow the development of pancreatic islet cell insufficiency by more than 50% while not causing an unacceptable increase in adverse.

In an alternative embodiment, activation of ngn3 and/or NeuroD/β2 gene expression is achieved through the administration of nucleic acid molecule encoding for ngn3 and/or NeuroD/β2 to a patient in a therapeutically effective amount where by “therapeutically effective amount” is meant an amount of the nucleic acid molecule sufficient to obtain the desired physiological effect, e.g., generation or restoration of, for example, insulin-secreting β-cells.

Screening Methods

In another aspect, the invention features an assay system for identifying compounds for the ability to activate ngn3 and/or NeuroD/β2 gene expression. In an illustrative embodiment, the isolated and proliferated progenitor cells, and their progeny, can be used to screen candidate compounds. In one embodiment of the method of identifying compounds that activate ngn3 and/or NeuroD/β2 gene expression, cells are incubated with and without the test compound, and the ability of the test compounds to activate ngn3 and/or NeuroD/β2 gene expression is measured. In one embodiment, the ability of the test compounds to activate ngn3 and/or NeuroD/β2 gene expression is indicated by the generation of insulin-secreting cells from the treated cells relative to the control untreated cells where the generation of insulin-secreting cells may be demonstrated by, for example, detection of the presence of RNA transcripts for β-cell associated differentiation markers such as synaptophasin, chromogranin A, prohormone convertase PC1/3, glucokinase or insulin. In a preferred embodiment, the cells to be treated with the test compound are precursor stem cells or adult duct cells.

In a related aspect, the invention features an assay system for identifying compounds with specifically in inhibit ngn3 and/or NeuroD/β2 expression. Such compounds are useful, for example, in in vitro culture systems for inhibiting premature differentiation of precursor stem cells into pancreatic cells.

Role of Neurogenin 3 in the Independent Development of Pancreatic α- and β-Cells

It has been thought that all islet endocrine cells would be related in a direct lineage relationship. However, more recent studies in transgenic mice that used the glucagon (GLU) or insulin (INS) promoter to drive expression of the diphtheria-toxin A-chain contradict this model. The lack of either α- or β-cells in these mice may mean that cells derive independently. Moreover, early appearing GLU⁺ cells do not divide, which makes it difficult to envision a significant contribution from these to the vast increase in β-cell mass seen during the secondary transition. More recent immunohistochemical analyses suggest that β-cells arise directly from the proto-differentiated epithelial cells, which express the β-cell markers glucose transporter type 2 (GLUT2), nerve growth factor receptor (TrkA), pancreatic duodenal homeobox factor (PDX1), and NK homeobox 6 (Nkx6.1) (Jonsson et al. (1994) Nature 371:606-609; Offield et al. (1996) Development 122:983-995; Øster et al. (1998) J. Histochem. Cytochem. 46:707-715; Pang et al. (1994) Proc. Natl. Acad. Sci. USA 91:9559-9563; and Scharfmann et al. (1993) Diabetes 42:1829-1836). However, because these cells also give rise to the acinar cells, the signals that determine the endocrine fate remains unknown.

Studies of pancreas development in mice deficient for Isl1 have hinted at a transcription factor cascade that operates in endocrine differentiation. Isl1^{-/-} mice lack Pax6 expression but retain NeuroD expression (Edlund (1998) Diabetes 47:1817-1823), which suggests that NeuroD acts upstream and Pax6 acts downstream of Isl1. Further clues to factors acting upstream of NeuroD emerge from studies of neural development. The neurogenin family of bHLH factors is thus required for expression of NeuroD family factors in certain neuronal subtypes (Ma et al. (1998) Neuron 20:469-482 and Fode et al. (1998) Neuron 20:483-494).

A study was conducted to determine the role of the bHLH gene in differentiation of β-cells (Examples 1-4 and Jensen et al. (2000) Diabetes 49:163-176). It was found that the bHLH gene Neurogenin3/MATH4B/Atoh5 (ngn3) is expressed in a subset of the proto-differentiated PDX1⁺ epithelial cells. Ki-67, a marker of proliferating cells, is readily detected in ngn3⁺ cells but not in NeuroD⁺ cells. A few ngn3⁺ cells co-express early endocrine markers, such as Isl1 and Pax6, whereas no ngn3⁺ cells co-express GLU or INS.

In contrast, it was found that NeuroD is co-expressed with Pax6 and glucagon or insulin. However, a few NeuroD⁺ cells do not express Pax6, which suggests that NeuroD is the earliest marker of differentiating endocrine cells. Surprisingly, the existence of a temporal sequence of gene activation and inactivation that differs between α- and β-cells was found. Thus, a molecular pathway where ngn3⁺ precursor cells become committed to endocrine differentiation is marked by activation of NeuroD and subsequently Isl1, Pax6, and the hormones, whereas ngn3 expression is extinguished. The choice between α- and β-cell fate appears to depend on a transcription factor code where Brain4/Pou3f4 (Brn4) determines α-cell development and Pdx1, Nkx6.1, and Pax4 determine β-cell development. Expression of Notch1 in the epithelial cells suggests that the choice between differentiation and continued proliferation is controlled by a Jagged-Notch-HES1 pathway that inhibits ngn3 function.

Using double immunofluorescence staining for glucagon and insulin together with the nuclear markers Pdx1, Isl1, Pax6, and Brn4, the development of the earliest endocrine cells in the mouse pancreas at the budding stage was analyzed (Example 3). The results showed that both the dorsal and ventral pancreatic bud are clearly defined at E10 by Pdx1 immunoreactivity. The dorsal bud is larger, contains more Pdx1⁺ cells, and is more mature than the ventral bud because GLU⁺ cells are found in only the dorsal bud. Most glucagon cells are devoid of Pdx1, but some Pdx1⁺/GLU⁺ cells are found, suggesting that the GLU⁺ cells derive from the Pdx1⁺ cells and subsequently lose Pdx1 expression. The GLU⁺ cells at this stage are mitotically quiescent, as determined by the lack of BrdU incorporation, and none express insulin at significant levels (data not shown). Evidence for a temporal sequence of gene activation comes from GLU/Pax6 and Pax6/Isl1 double immunofluorescent stainings. GLU⁺ cells invariably express Isl1 (data not shown) and Pax6. However, the presence of GLU⁻/Pax6⁺ and Pax6⁻/Isl1⁺ cells suggests that Isl1 expression precedes Pax6, which precedes glucagon production.

A RT-PCR screening was performed (Example 4 below) for atonal-like bHLH factors in various stages of embryonic pancreas and found that ngn3 and NeuroD were strongly expressed (data not shown), which is in agreement with previously published results. The spatial pattern of ngn3 and NeuroD expression was determined by whole-mount in situ hybridization. At the earliest stage examined (E9.5), ngn3 was found to be expressed in a speckled pattern in the dorsal pancreatic bud exclusively. A similar profile, though with increased expression, was observed at E10.0. At E13.5 and E15.5, expression was evident in both the dorsal and ventral pancreas. Through stages E9.5-E15.5, an increase in the expression level of ngn3 was observed. RT-PCR analysis confirmed the increase in ngn3 expression until E15 (data not shown). At E17, only a small part of the epithelium showed detectable ngn3 expression and at a low intensity (data not shown). Pancreatic expression of NeuroD examined by whole-mount in situ hybridization was superficially similar to the ngn3 expression. To determine the cellular localization of ngn3 and NeuroD expression, the whole-mount in situ hybridized embryos were sectioned and immunohistochemical stainings performed with antisera against Pdx1, Pax6, and glucagon.

At E10.0, the ngn3⁺ cells were found in clusters and co-localized with a subset of the Pdx1⁺ cells. In contrast, ngn3 expression in Pax6⁺ and GLU⁺ cells was low or absent. At E13.5, Pax6⁺ cells were mostly outside the ngn3⁺ domain. However, a few Pax6⁺ cells were clearly coexpressing ngn3, which suggests that endocrine cells derive from the ngn3⁺ cells and subsequently lose ngn3 expression. In contrast, Pax6⁺ cells (GLU⁺ and GLU⁻) also express NeuroD. Occasionally, it was found that NeuroD⁺ cells that did not express Pax6, which supports the notion that NeuroD acts upstream of Isl1 and Pax6. A similar profile was observed at the peak of ngn3 expression (E15.5), when Pax6⁺ cells (with a few exceptions) were outside the ngn3⁺ domain and GLU⁺ and Ins⁺ cells are excluded from the ngn3⁺ domain. At E17.5, when ngn3 expression was found only in a small part of the epithelium, we again found that Pax6⁺ and Ins⁺ cells are excluded from the ngn3⁺ domain, but they overlap with the NeuroD⁺ domain. GLU⁺ cells also were excluded from the ngn3⁺ domain at this stage, whereas Nkx6.1^{low} cells can be found in some but not all ngn3⁺ cells. In contrast, Nkx6.1^{high} cells are always outside the ngn3⁺ domain, but inside the NeuroD⁺ domain. At all stages examined, we found endocrine cells located at close proximity to the ngn3⁺ cells, which is consistent with a role for the ngn3⁺ cells as precursors for the endocrine cells. Ngn3 expression was never observed in the pancreatic acinar cells at E15.5 or later.

To examine whether ngn3⁺ cells were proliferating, we stained sections of ngn3 whole-mount in situ hybridizations for Ki-67, a marker of proliferating cells. Ki-67⁺ nuclei are present within the ngn3⁺ domain, but absent from the NeuroD⁺ domain. The lack of BrdU incorporation in E14 Isl1⁺ cells corroborates the postmitotic nature of the endocrine cells at this stage. In contrast, endocrine cells are proliferating at E19, as visualized by BrdU incorporation in Isl⁺ cells. Consistent with previous studies (Hellerstrom (1976) Acta Endocrinol Suppl (Copenh) 205:145-160), we found that both α- and β-cells were proliferating at E19, as seen by BrdU incorporation into GLU⁺ and Nkx6.1⁺ cells.

Independent Formation of α- and β-Cells from Epithelial Precursor Cells at the Secondary Transition.

The issue of the origin of the pancreatic β-cells is controversial. A study was conducted to analyze the development of endocrine cells at the secondary transition by determining the expression pattern of transcription factors that precede the expression of hormones and are common to all endocrine cells, and transcription factors specific to α- or β-cells.

First, Isl1 and Pax6 expression was studied at E14. It was found that all Pax6⁺ cells co-expressed Isl1, which was similar to the situation at E10. However, the presence of Isl1⁺/Pax6⁻ cells were evident also at this stage, suggesting that endocrine cell development from hormone negative cells occurs at this stage. In an attempt to discriminate between α- and β-cells, the expression of the α-cell marker Brn4 was analyzed in relation to Isl1, glucagon, and insulin. The results showed that Isl1⁺ cells can be divided into a Brn4⁺ and a Brn4⁻ population. Because we found expression of Brn4 in all glucagon-producing cells, the presence of Brn4⁺/Isl1⁻ cells is best explained by activation of Brn4 expression before Isl1⁻ and, subsequently, glucagon expression in α-cell development.

The validity of this hypothesis was tested by finding Brn4⁺/GLU⁻ cells at the E14 stage. The Brn4⁻/Isl⁺ population most likely represents β-cells that express Isl1 but not Brn4. Brn4⁺ cells did not incorporate BrdU during a 90-min pulse, which indicates that they are postmitotic. Whereas most GLU⁺ cells were Pdx1⁻ at E14, we did see scattered GLU⁺ cells that were PDX1⁺. However, cells that expressed high levels of Pdx1 (Pdx1^{high}) were not found to co-express glucagon at any stage.

Pdx1^{high} cells were often found to be Ins⁺ at E14. However, Pdx1^{high}/Ins⁻ cells were also found at E14, suggesting that upregulation of Pdx1 precedes Ins production. The Pdx1^{high} cells did not incorporate BrdU, whereas intermediate-level Pdx1⁺ cells did incorporate BrdU. Consistent with the absence of BrdU in Pdx1^{high} cells and with the Ins⁺/Pdx1^{high} co-expression, Ins⁺ cells were never found to incorporate BrdU at E14.

Dynamic Expression of Notch-1 and -2 During Pancreas Development.

Mammalian homologs of the Drosophila hairy- and enhancer-of-split factors (HES proteins) are negatively acting bHLH proteins that antagonize members of the Ngn and NeuroD family. Hes1 was the only member of the HES family that was detected in an RT-PCR screening for expression of these factors in the embryonic pancreas. Hes1 mutant animals display pancreatic hypoplasia due to accelerated differentiation of endocrine cells that results in a depletion of epithelial precursors. As Hes1 is a known downstream component in the Notch-signaling system, we sought to identify if any of the mammalian Notch-factors were expressed during pancreatic development. We found that three mammalian Notch genes, Notch-1, -2, and -3, but not Notch-4, were expressed during pancreatic development. In situ hybridization on sections of embryonic pancreas revealed that Notch-1 is expressed in the epithelium at E12. At E15.5, Notch-1 mRNA is located in the forming exocrine tissue. At E17.5, localization of both Notch-1 and -2 in exocrine tissue and absence from endocrine tissue was evident. Notch-1 is also absent from most endocrine cells at E13.5, although a few Pax6⁺ cells are located still in the Notch-1⁺ Similar to the mouse, E13 rat pancreas expresses Notch-1 and -2 and Jagged1 primarily in the epithelial cells and significantly in the mesenchyme. RT-PCR analysis of microdissected rat embryonic pancreas revealed that Notch-1, -2, and -3 are expressed at increasing levels until E15.5 and followed by a marked decline in the next 2 days. A second phase of high expression occurs after the secondary transition. The decline in Notch gene expression appears at the onset of the secondary transition and is marked by increased expression of insulin and amylase. Furthermore, the increase in NeuroD and p48-PTF1 gene expression is similarly observed between E16.5 and E17.5. In light of the expression of the Notch ligands Dll1 in a few pancreatic epithelial cells and Jagged1 in most pancreatic epithelial cells, the Notch-1 expression pattern at this stage supports Notch-1 transducing a signal that counteracts endocrine differentiation.

Role of Ngn3 and NeuroD/β2 in the Transdifferentiation of Pancreatic Exocrine Duct Cells into Insulin Secreting Cells

The plasticity of the adult exocrine pancreas has been demonstrated extensively. Acinar cells transdifferentiate in vitro into hepatocytes {Shenet al. Nat. Cell. Biol. 2: 879-887 (2000)} as well as into exocrine duct cells {Roomanet al. Diabetologia 43: 907-914 (2000)}. Moreover, endocrine islet cells have been proposed to arise from the pancreatic exocrine duct cells {Slack Development 121: 1569-1580 (1995)} {Bouwens & Pipeleers 1998 #213} {Bonner-Weiret al. Proc. Natl. Acad. Sci. USA 97:7999-8004 (2000)} although there is no direct evidence for this concept. As the number of donor islets is far insufficient for treatment of all diabetic patients but the abundance of exocrine cells is high and can even be expanded {Lefebvreet al. Diabetes 47: 134-1 37 (1998)}, a demonstration of the endocrine differentiation capacity of pancreatic exocrine cells by transgenic complementation of the exocrine cells with an essential master switch would be of high clinical relevance for islet transplantation.

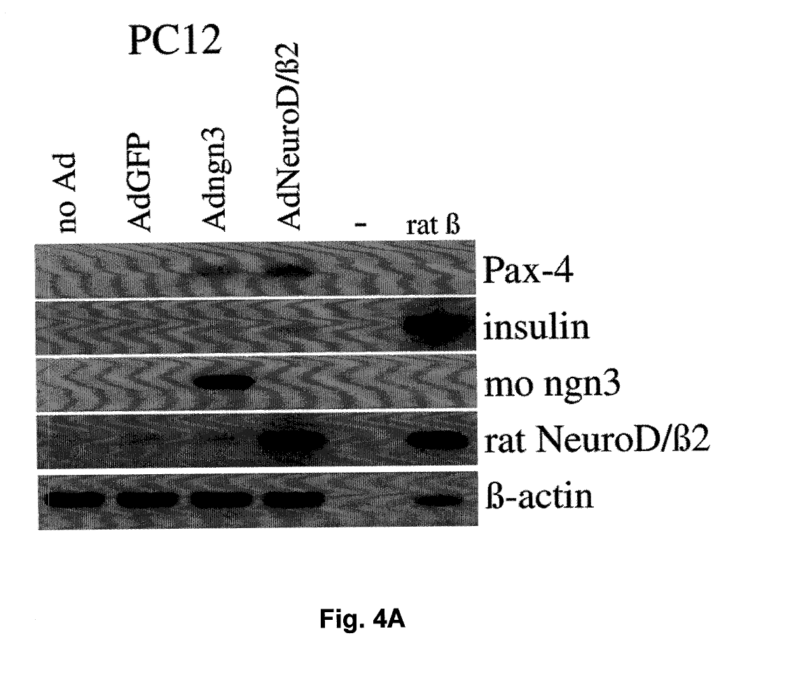

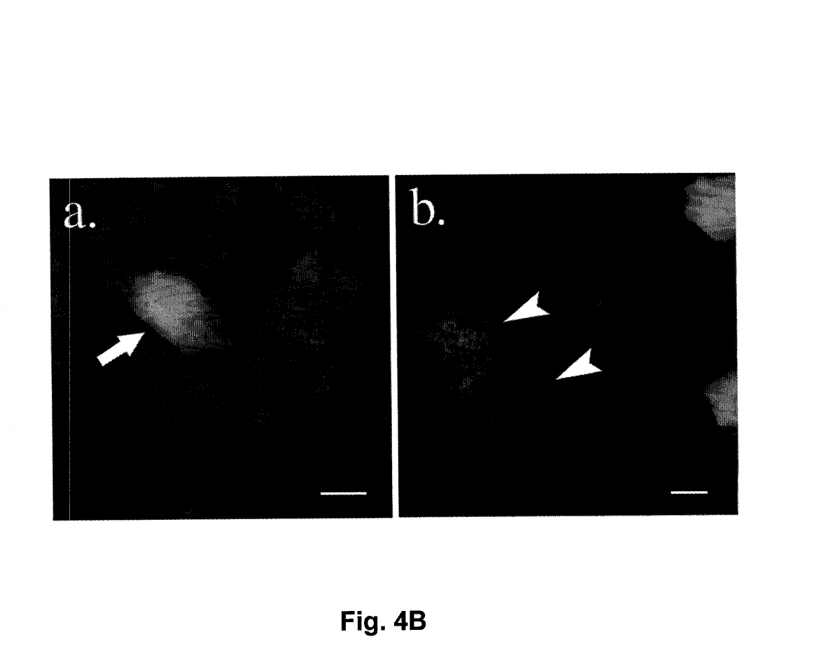

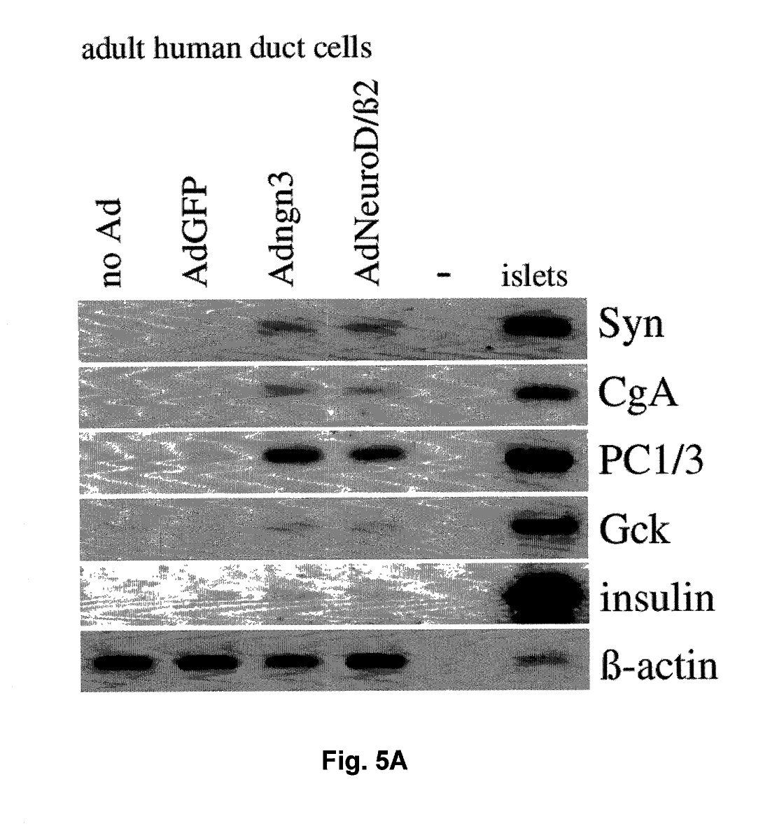

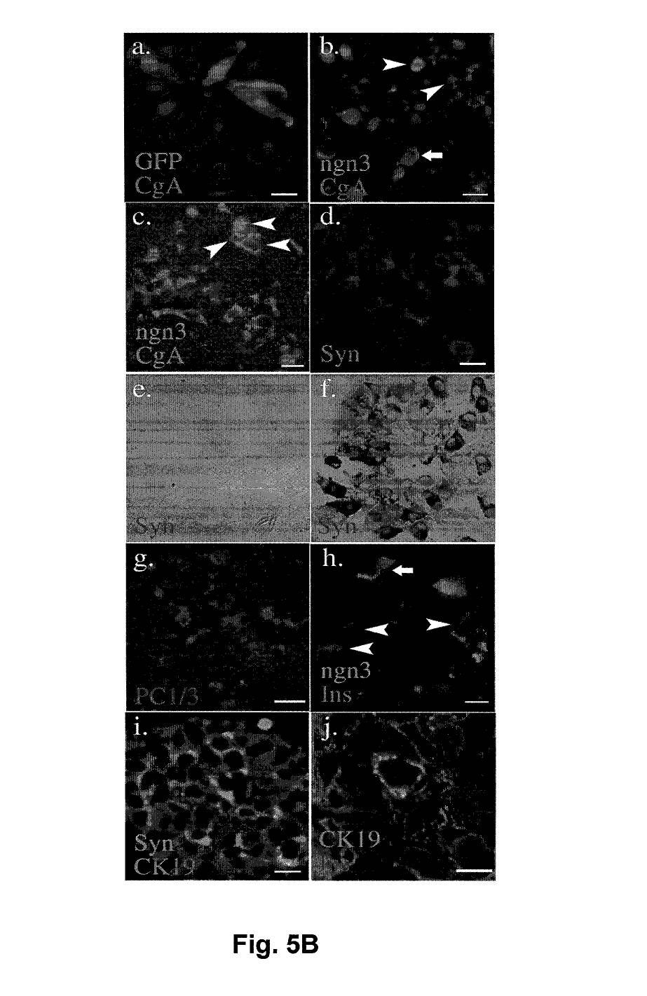

Here in the present application, it is demonstrated that primary adult human duct cells are an amenable and abundant cell source for in vitro differentiation towards β-cells. Specifically, the results presented in Example 5B demonstrate the feasibility of β-cell neogenesis from adult human exocrine cells by adenovirus-mediated delivery of developmental transcription factors that control pancreas embryogenesis. In particular, ectopic ngn3 or NeuroD/β2 activated numerous genes encoding endocrine-specific proteins in adult human duct cells (see FIG. 5A) and the observed effects were cell type-restricted as they could be reproduced in the clonal neuroendocrine cell line PC12 but not in HeLa cells (FIG. 4A and data not shown).Thus, the results presented in Example 5B demonstrate the unique plasticity of the duct cell. Further, since no intentional selection was made among the duct cells during their processing, all of the duct cells apparently exhibited equal sensitivity to ngn3- and NeuroD/β2-induced transdifferentiation.

Based on the phenotypes of transgenic mice a hypothetical hierarchy of transcription factors —Pdx1/Ipf1>ngn3>NeuroD/β2>Pax4>Nkx2.2>Nkx6.1—leading to embryonic formation and differentiation of insulin-positive β-cells has been proposed {Schwitzgebelet al. Development 127: 3533-3542 (2000)}. The results presented in Example 5B provide direct experimental evidence for this model, showing that recapitulation of endocrine differentiation in differentiated human duct cells obeys the same hierarchy. Thus, induction of differentiation by the principle of lateral inhibition {Artavanis-Tsakonaset al. Science 284: 770-776 (1999)}, common in embryonic development of many tissues among which the endocrine pancreas {Apelqvist, et al. (1999) Nature 400:877-81} {Jensen, et al. Diabetes 49: 163-176 (2000)} is achievable by ectopic expression of ngn3 or NeuroD/β2 in adult human duct cells. Moreover, our results demonstrate that Pdx1/Ipf1 is not indispensable for the ngn3 effects and that Nkx6.1 gene activation and mRNA translation is no immediate consequence of ngn3 or Nkx2.2 expression. Adenovirus-mediated expression of recombinant transcription factors in adult human duct cells is thus not only a method for stimulating β-cell neogenesis from adult human exocrine cells but can also serve as a simple and highly valuable supplement to the existing transgenic mice models for studying the molecular biology of endocrine differentiation.

Finally, while infection of adult human duct cells with ngn3 or NeuroD/β2 induced the expression of the insulin gene as well as a broad variety of markers of the differentiated (neuro)endocrine phenotype-synaptophysin {Wiedenmann, et al. Proc. Natl. Acad. Sci. USA 83: 3500-3504 (1986)}, chromogranin A {Kim et al. Cell 106: 499-509 (2001)}, PC1/3 {Marcinkiewicz et al. Endocrinology 135: 1651-1660 (1994)} and glucokinase {Grupe, et al. Cell 83: 69-78 (1995)}, the abundance of insulin mRNA and protein in Adngn3- or AdNeuroD/β2-infected adult human duct cells may be increased by any one of the following approaches: (i) since Nkx6.1 is essential for differentiation of insulin-producing β-cells {Sanderet al. Development 127: 5533-5540 (2000)} but is neither endogenouslypresent at protein level nor ngn3-induced in monolayers of adult human duct cells, superinfection of Ngn-3-expressing duct cells with an AdNkx6.1 vector containing a truncated Nkx6.1 cDNA (where the truncated cDNA is missing the sequence in the 5′-untranslated region of the Nkx6.1 transcript that has been identified as involved in translational control; Jorgensen et al 1999 FEBS Letters 461: 287-294), may thus increase insulin production. (ii) since Pax4 is reported to have an inhibitory action on insulin gene expression {Smithet al. Mol. Cell. Biol. 19: 8272-8280 (1999)} {Campbell, et al. FEBS Lett 463: 53-57 (1999)},the persistent elevation of ngn3, and consequently Pax4, in Adngn3-infected cells may suppress the number of insulin-positive duct cells. The above hypothesis is supported by the fact that ngn3-induced insulin gene expression in fast dividing PC12 cells was high. Thus, conditional expression of ngn3 in adult human duct cells, mimicking its transient embryonic expression, may augment insulin gene expression, where conditional expression is achieved through the use of expression systems in which ngn3 gene expression is under the control of an inducible or repressible promoter system. An example of a repressible system is the Adeno-X Tet-Off Gene Expression System® (Clontech) where gene expression is shut off by exposure to tetracycline or a tetracycline analog. An example of an inducible system is the tamoxifen inducible expression ogf a hybrid of the ngn3 gene and a mutated steroid receptor. (iii) infecting cells with transcription factors that are located downstream of ngn3 and are able to induce differentiation towards beta cells without inducing Dll1 since neurogenin-3 was observed to activate transcription of Dll1 and Dll4, thus allowing increased Notch signaling in the neighbouring duct cells, which might result in an overall decrease of endocrine differentiation (iv) treatingn3 and/or NeuroD/β2-infected cells with additional pancreatic factors such as activin, gastrin or glucagon-like peptide 1 (GLP-1).

### EXAMPLES

The following examples are put forth so as to provide those of ordinary skill in the art with a complete disclosure and description of how to generate mature pancreatic cells from precursor stem cells, as well as how to identify compounds which activate or inhibit neurogenin 3 gene expression, and therapeutic methods for treatment or prevention of pancreatic islet cell insufficiency, and are not intended to limit the scope of what the inventors regard as their invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Animals and Pancreas Microdissection

Time-mated pregnant mice (CD1) and rats (Wistar-Furth) were obtained from Bomholtgåard Breeding Centre (Ry, Denmark). Embryos (one litter at a time) were liberated in chilled Hanks' balanced salt solution; pancreases were collected by microdissection and were transferred directly to RNAzol-B (Biotecx, Houston, Tex.) for reverse transcriptase-polymerase chain reaction (RT-PCR) analyses. Material used for histological analysis was fixed overnight in 4% paraformaldehyde (PFA) before paraffin-embedding. Sections of 4 μm were cut and stored at room temperature until use. Material for whole-mount in situ hybridizations was fixed overnight in 4% PFA in Ca²⁺ and Mg²⁺ free phosphate-buffered saline (PBS) and 2 mmol/l EGTA. The tissues were dehydrated in methanol and stored at −20° C. until use. For RT-PCR, rat pancreatic tissue originating from the dorsal bud was isolated before fusion; at later stages, tissue from the prominent splenic portion, which was also dorsally derived, was used. At the earliest time points (E12.5 and E13.5), a full litter (˜10-12) of isolated pancreatic buds was combined due to the small amount of tissue. At later stages, fewer pancreases were used, and after El 7.5, isolations were performed on material from a single organ. No separation of the pancreatic mesenchyme was performed. However, at later stages, the portion of the mesenchyme that was condensed to form the spleen and that was visible during E14-16 was removed. At least two independent cDNA preparations from the different time points were analyzed (except at E12.5).

### Example 2

### Antisera and in situ Hybridization Probes

Guinea pig anti-insulin was obtained from Nordisk Gentofte A/S (Gentofte, Denmark). Mouse monoclonal antiglucagon-Glu001 was obtained from Novo Nordisk A/S (Bagsværd, Denmark). Rabbit anti-PDX1-1856 and rabbit anti-Nkx6.1-174 have been described previously (Jensen et al. (1996) J. Biol. Chem. 271:18749-18758). Rabbit anti-IDX1-253 (Stoffers et al. (1998) J. Clin. Invest. 102:232-241) was a gift from J. Habener. Rabbit anti-BRN4 was a kind gift from M. Rosenfeld. Rabbit anti-PAX6-Bg11 (Turque et a. (1994) Mol. Endocrinol. 8:929-938) was a gift from S. Saule. Rabbit anti-ISL1 (Thor et al. (1991) Neuron 7:881-889) was a gift from H. Edlund. Mouse monoclonal anti-ISL1, developed by T. Jessell, was obtained from the Developmental Studies Hybridoma Bank, which is under the auspices of the National Institute of Child Health and Human Development and maintained by the Department of Biological Sciences at the University of Iowa (Iowa City, Iowa). Mouse monoclonal anti-5-bromo-2′-deoxyuridine (BrdU)-Bu20A was from DAKO A/S (Glostrup, Denmark). Mouse monoclonal anti-Ki-67-B56 was from Pharmingen (San Diego, Calif.). Plasmids for synthesis of digoxigenin-labeled riboprobes, such as mouse ngn3, rat NeuroD, and rat Notch-1 and -2, were donated by F. Guillemot, F. G. Andersen, and J. Hald, respectively. The ngn3 plasmid was cut with HindIII and transcribed with SP6 RNA polymerase; the NeuroD plasmid was cut with BamHI and transcribed with T7 RNA polymerase; the Notch-1 and -2 plasmids were cut with BamHI and SpeI, respectively, and transcribed with T7 RNA polymerase in the presence of digoxigenin-UTP (Boehringer Mannheim, Mannheim, Germany).

### Example 3

### Immunohistochemistry and in situ Hybridization

Imunohistochemistry was performed as described in Blume et al. (1992) Mol. Endocrinol. 6:299-307. Briefly, sections were dewaxed in xylene and rehydrated through a descending ethanol series. Antigen retrieval was accomplished through microwave treatment (two times for 5 min at 600 W in 0.01 mol/l citrate buffer, pH 6.0) followed by three washes in PBS. Nonspecific binding was blocked with 10% donkey nonimmuneserum. For double immunofluorescence, sections were incubated with primary antibodies overnight. Secondary antibodies (fluorescein isothiocyanate-, Cy-2-, and Texas-Red-conjugated) were obtained from Jackson ImmunoResearch (West Grove, Pa.). In situ hybridization on paraffin sections was performed by first dewaxing and rehydrating paraffin sections through an ethanol series. Three washes in diethyl pyrocarbonate (DEPC)-treated PBS were followed by a 5-10 min treatment with proteinase K (10 μg/ml). Slides were fixed in fresh 4% paraformaldehyde and rinsed in DEPC PBS. Sections were acetylated for 10 min and permeabilized with Triton X 100 (1%) for 30 min. Prehybridization was for 2 h, and slides were incubated with digoxigenin-labeled antisense riboprobes overnight at 72° C. in a humidified chamber. The unhybridized probe was removed with subsequent rinses in 5× sodium chloride-sodium citrate (SSC) and 0.2× SSC at 72° C. for 1 h each. Slides were rinsed in Tris-buffered saline with Tween with 2 mmol/l Levimasole (Sigma, St. Louis, Mo.) and blocked with 10% heat-inactivated sheep serum. Alkaline phophatase-conjugated Fab fragments (Boehringer Mannheim), preabsorbed with E17 embryo powder, were added to the slides overnight at 4° C. Slides were rinsed in NTMT (0.1 mol/l NaCl, 0.02 mol/l Tris [pH 9.5], 0.01 mol/l MgCl₂, and 1% Tween-20) three times for 10 min, and the reaction mixture was added (nitroblue tetrazolium and 5-bromo-4-chloro-3-indolylphosphate (Boehringer Mannheim) in NTMT). Reactions were allowed to proceed for 2 h to 5 days at room temperature. The reaction was stopped by several rinses in Tris-EDTA, and slides were mounted and coverslipped. Whole-mount in situ hybridizations on mouse embryos were performed as previously described (Hume et al. (1993) Development 119:1147-1160). Subsequently, the whole-mount specimens were cryo-protected in 30% sucrose in 0.1 mol/l phosphate buffer overnight and embedded in Tissue-Tek (Sakura Finetek Europe, Zoeterwoude, the Netherlands), and 10-μm sections were cut on a cryostat. Sections were frozen and stored at −80° C. Images were collected by using an Olympus BX60 microscope (Olympus Optical, Tokyo) equipped with a Hamamatsu-chilled C5810 Color CCD camera (Hamamatsu Photonics, Solna, Sweden), and images were processed using the IMAGE-Pro PC software package (Media Cybernetics, Silver Spring, Md.) and Adobe Photoshop 3.0 (Adobe Systems, San Jose, Calif.). False color images were generated in Adobe Photoshop 3.0 by using the invert and selective color algorithms.

### Example 4

### RNA Isolation, cDNA Synthesis, and Multiplex RT-PCR

RNA was isolated from the microdissected embryonic tissue using the RNAzolB single-step method (Biotecx). Tissue was transferred to chilled eppendorfs containing 100 μl RNAzol-B and were homogenized using motor-driven micropistils (Eppendorf, Hamburg, Germany). Chloroform (10 μl) was added, followed by vortexing and 5′ centrifugation at 10,000 rpm. The aqueous phase was precipitated using 1 volume isopropanol, and subsequently reprecipitated by 2.5 volumes of ethanol and 0.1 volume of 4 mol/l NaCl. Total RNA pellets were dissolved using DEPC-treated water. cDNA synthesis was performed on 1 μg total RNA as described in a previous study (Jensen et al. (1996) supra). Multiplex RT-PCR was performed as described by Jensen et al. (1996) supra), with the following modifications. The reaction volume was lowered to 25 μl. All final concentrations of buffers, dNTPs, and primers were unchanged. The template concentration was decreased accordingly (the template per reaction was 1/50th of the cDNA reaction performed on 1 μg total RNA). All reactions were performed in 0.2-ml microtube strips (Stratagene) or 96-well plates, and mineral oil was included during cycling. The thermal-cycling profile was set at 1′ denaturation at 96° C., followed by the selected number of cycles: 30 s at 96° C., 30 s at 55° C., and 30 s at 72° C. The increase in denaturing temperature has shown an improved amplification of G/C rich amplicons. All pipetting steps in the RT-PCR analyses were performed using a multichannel pipette. To increase accuracy, no pipetting volumes were set below 3 μl. Sequencing gel runs were performed as described by Jensen et al. (1996) supra. For neurogenin-3, 160 bp, the primer sequence (upstream primer listed first) was 5′-TGGCGCCTCATCCCTTGGATG-3′ (SEQ ID NO:1), 5′-CAGTCACCCACTTCTGCTTCG (SEQ ID NO:2) (Genbank no. U76208); for HES-1, 270 bp, the primer sequence was 5′-TCAACACGACAC-CGGACAAACC-3′ (SEQ ID NO:3), 5′-GGTACTTCCCCAA-CACGCTCG-3′ (SEQ ID NO:4) (Genbank no. D13417); for p48/PTF-1, 249 bp, the primer sequence was 5′-AAGGTCATCATCTGCCATCGAG-3′ (SEQ ID NO:5), 5′-AGCCGG-CCTGTGAGAGCTTTC-3′ (SEQ ID NO:6) (Genbank no. X98446); for MASH-1, 220 bp, the primer sequence was 5′-GTCGAATACATCCGCGCGCTG-3′ (SEQ ID NO:7), 5′-AGAACCAGTT-GGTAAAGTCCA-3′ (SEQ ID NO:8) (Genbank no. X53725); for amylase, 300 bp, the primer sequence was 5′-GAGGACATGGTGCTGGAGGAG-3′ (SEQ IS NO:9), 5′-CGTTGACTA-CATTCCTGAAGG-3′ (SEQ ID NO:10) (Genbank no. M24962); for c-Met, 230 bp, the primer sequence was 5′-TTATGGACCCAACCACGAGCAC-3′ (SEQ ID NO:11), 5′-GAAGCGACCTTCTGATGTCCC-3′ (SEQ ID NO:12) (Genbank no. 69881); for Isl-2, 272 bp, the primer sequence was 5′-GCAGCCAGTATCTGGATGAGAC-3′ (SEQ ID NO: 13), 5′-CTCCAGCAGGAGGCCGTGGT-3′ (SEQ ID NO:14) (Genbank no. L35571); for Notch-1, 170 bp, the primer sequence was 5′-CTGGTTCCCTGAGG-TTTCAA-3′ (SEQ ID NO: 15), 5′-GGAACTTCTTGGTCTCCAGGT-3′ (SEQ ID NO: 16) (Genbank no.X57405); for Notch-2, 190 bp, the primer sequence was 5′-CAACATGGGCCGCTGTCCTC-3′ (SEQ ID NO: 17), 5′-CACATCTGCTTGGCAGTTGATC-3′ (SEQ ID NO: 18) (Genbank no. 93661); for Notch-3, 230 bp, the primer sequence was 5′-GCAGCTGTGAACAACGT-GAG-3′ (SEQ ID NO: 19), 5′-AACCGCACAATGTCCTGGTGC-3′ (SEQ ID NO:20) (Genbank no. X74760); and for Jagged-1, 270 bp, the primer sequence was 5′-AAGCCACGTGTAAT-ACGGTGG-3′ (SEQ ID NO:21), 5′-CTCCATCCACACAGGTTCCGC-3′ (SEQ ID NO:22) (Genbank no. L38483).

### Example 5

### In vitro Duct Cell Expansion and β-Cell Differentiation

The initial objective is to establish two defined cell culture media capable of supporting a high degree of growth and differentiation, respectively, of human pancreatic duct cells. The approach is based on part on access to primary human duct cells from the Eurotransplant program.

Initial target experiments identify candidate growth factors able to induce growth of primary human duct cells where the identification of such factors provides one with conditions that allow for the expansion in vitro of the population of duct cells that can be subjected to treatment with ngn3 or NeuroB/D2 as described below in Example 5B.

A second series of experiments define a serum free medium capable of inducing β-cell differentiation of human duct cells. Candidate differentiation factors are identified based on the ability to induce differentiation. Additionally, the fractionated extracts from IDR (AMP) are tested for the ability to induce proliferation and/or differentiation.

### Example 5A

### Identification of Candidate Growth Factors able to Induce Growth of Primary Human Duct Cells

As described above, the following study was carried out to determine conditions under which human postnatal duct cells are able to proliferate. In brief, the human pancreata exocrine fraction obtained as described in the Materials and Methods section for Example 5B was cultured for 4 days in suspension followed by transfer to human fibronectin coated dishes in the presence of EGF (25 ng/ml), KGF (25 ng/ml) or betacellulin (1 nM). One week after growth factor addition, the cultures were fixed, stained for cytokeratin 19 (duct cell marker) and Ki67 (S phase marker). In the absence of growth factors no duct cells express Ki67 whereas after KGF, betacellulin, and EGF treatment, 2%, 6% and 8% of duct cells were Ki67 positive, respectively (data not shown). Spreading of the cell clusters was also observed in the presence of EGF and betacellulin. By comparison, HGF, IGF, and FGF-10 induce minimal changes in proliferation.

In order to determine if MAP kinases are important mediators of the proliferation and differentiation observed in response to EGF, KGF and betacellulin, the status of different MAP kinase pathways was analyzed by western blots using phosphospecific antibodies. The results demonstrated that p38 MAP kinase and SAP kinases remain unphosphorylated after the addition growth factors (data not shown). On the contrary, phospho MEK1/2 and phospho ERK1/2 were detected rapidly (20 and 120 minutes) after EGF and betacellulin stimulation, but not detected in control or KGF-treated cultures (data not shown). A correlation between the amount of spreading, proliferation, and activation of the MEK/ERK pathway was identified. The role of MEK/ERK pathway was consequently assayed, via the use of the MEK inhibitor PD98059. PD98059 inhibited EGF-induced spreading of clusters. In addition, EGF-induced spreading was totally abolished when cells were infected with adenoviruses carrying a dominant negative form of MEK. To determine whether MEK/ERK activation was sufficient for spreading, cells were infected with an adenovirus carrying constitutive active MEK: In the absence of EGF, these cells spread in a similar manner as cells cultured in EGF. These data show that EGF and betacellulin can induce proliferation of a subset of human duct cells and modulation of the MEK/ERK pathway is critical for the expansion of human duct cells in vitro.

### Example 5B

### Identification of Transcription Factors Necessary To Induce β-Cell Differentiation of Duct Cells

A first in vitro culture of primary human duct cells was established. Secondary target experiments are then conducted to define the minimum complement of transcription factors necessary to induce β-cell differentiation of duct cells. The approach is to transfer candidate transcription factor cDNAs by adenovirus mediated gene transfer into human duct cells and analyse for differentiation markers.

Partial endocrine differentiation was induced by ngn3 expression.

Presented below are the experiments conducted to define the minimum complement of transcription factors necessary to induce β-cell differentiation of duct cells. The materials and methods utilized in these experiments are described in the section entitled “Materials And Methods” that follows the reporting of the results of these experiments. As described above, the approach utilized in the following experiments was to transfer candidate transcription factor cDNAs by adenovirus mediated gene transfer into human duct cells and analyse for differentiation markers.

Profiling the Expression of Markers for Development and Differentiation in Duct Cells of the Adult Human Pancreas.

The cells in this study were from donor pancreata processed for islet transplantation of type I diabetes patients. The endocrine preparations were more than 60% pure, and most of exocrine cells expressed the duct cell-specific phenotypic markers cytokeratin 19 and carbohydrate antigen 19.9 when cultured for at least 4 days {Bouwens, et al. Diabetes 43:1279-1283 (1994)}{Heimberg, et al. Diabetes 49: 571-579 (2000)}. Precursor cells of the endocrine pancreas are located among the cells lining the exocrine ducts {Slack Development 121: 1569-1580 (1995)} {Bouwens & Pipeleers (1998) Diabetologia 41:629-633}{Bonner-Weir, et al. (2000) Proc. Natl. Acad. Sci. USA 97:7999-8004} although it is not clear whether all duct cells or only a subpopulation of them sustain precursor cell capacity.

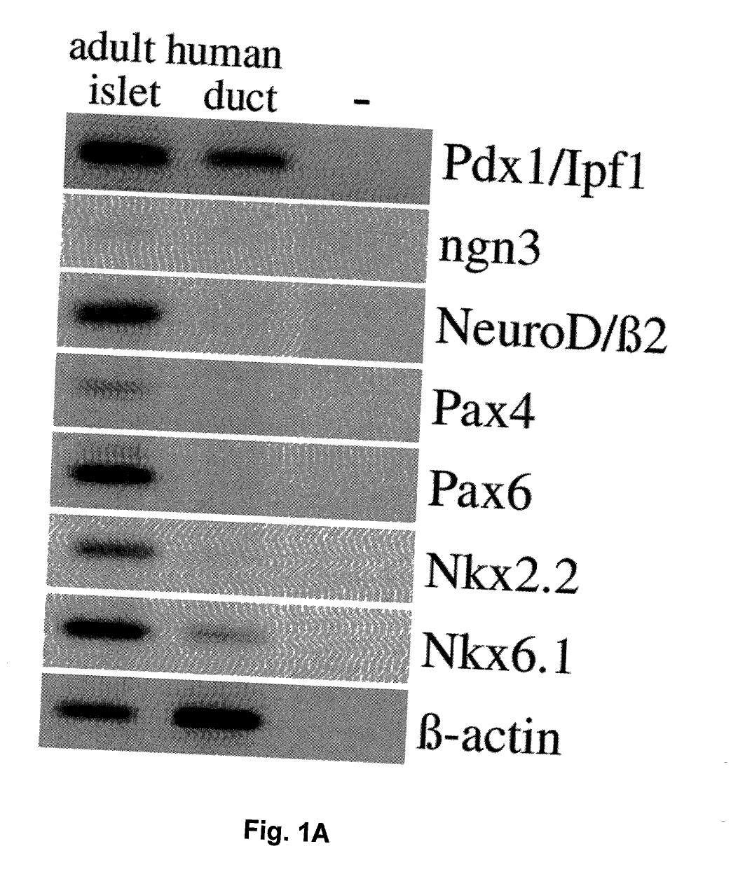

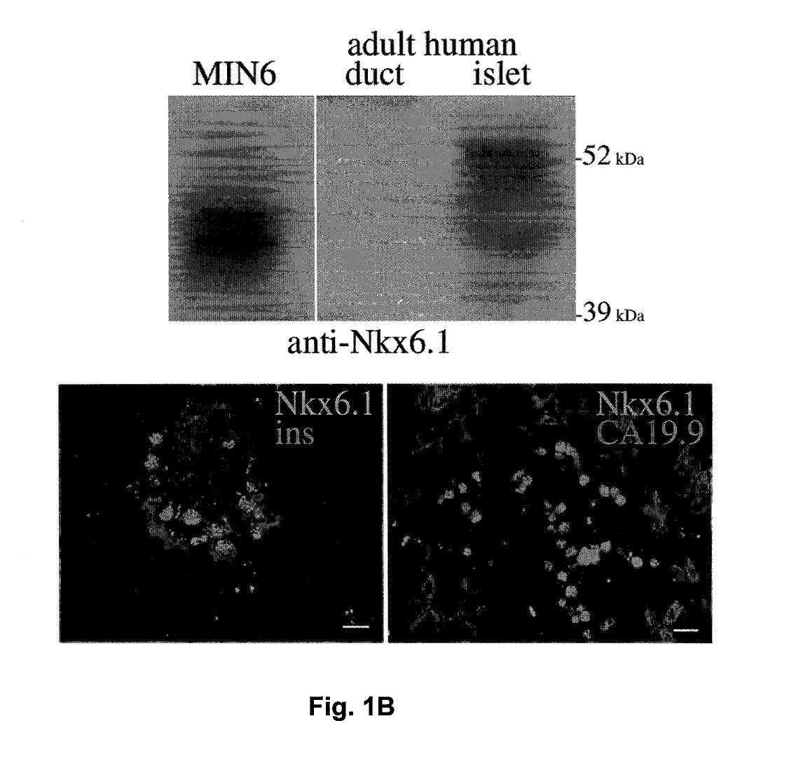

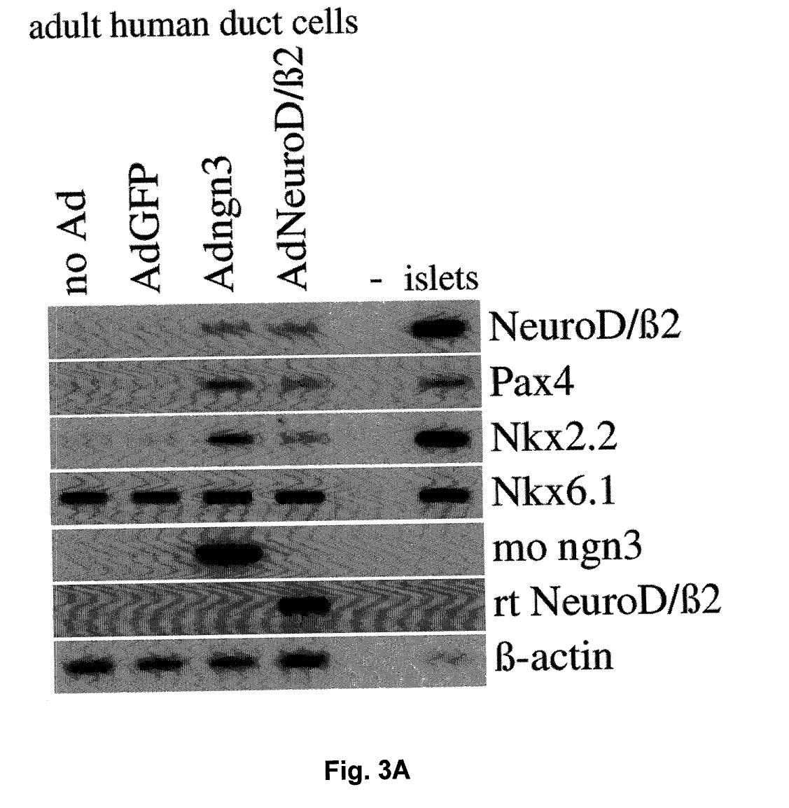

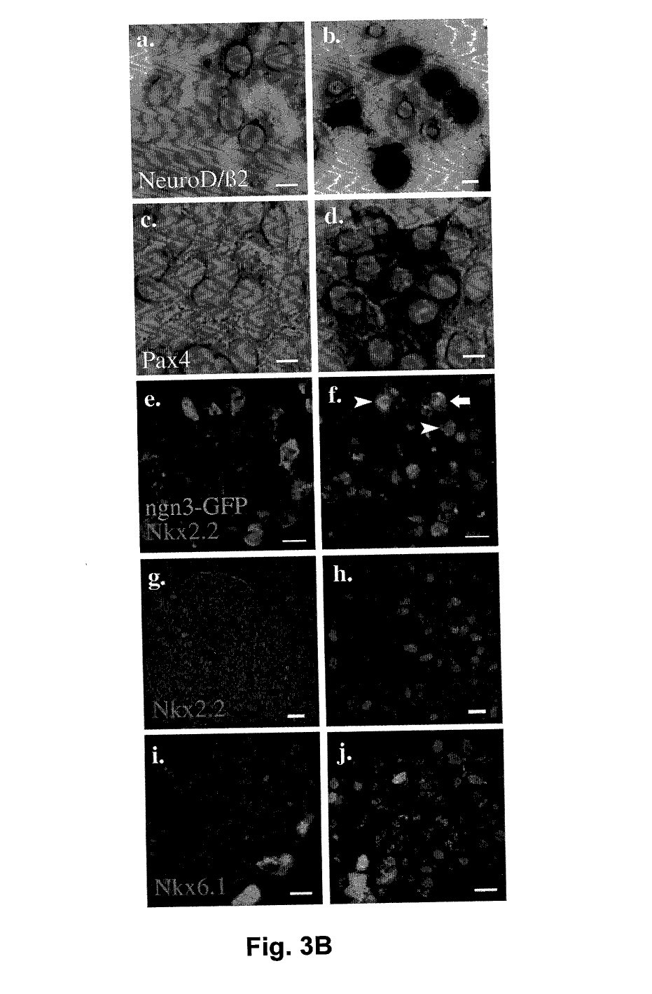

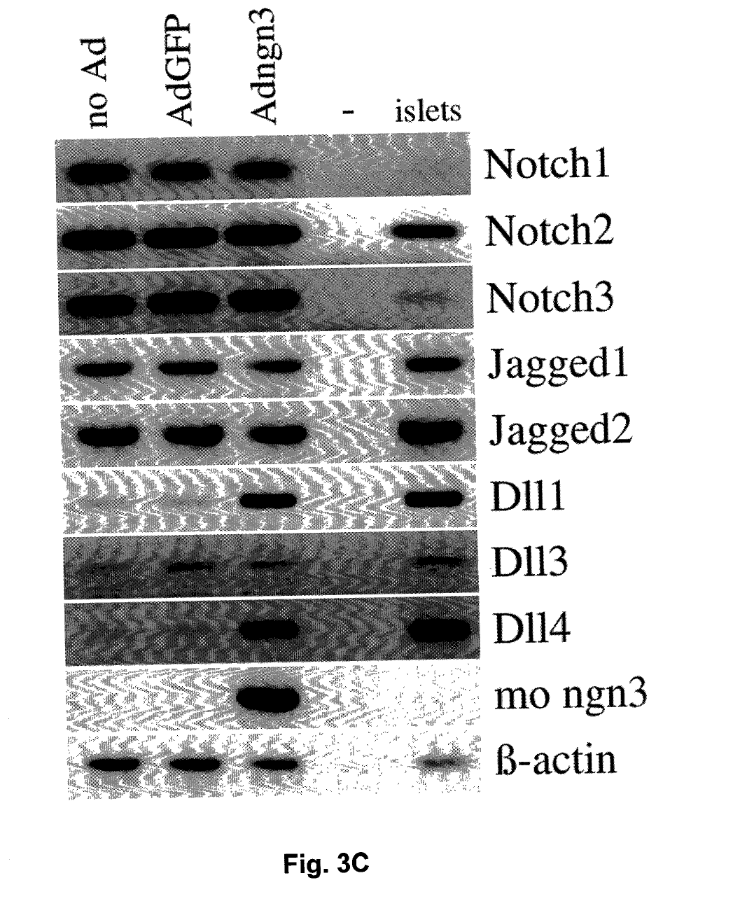

To emphasize differential gene regulation in mature islet and duct cells, the expression pattern of developmental transcription factors that are essential for the embryogenesis and maintenance of a fully functional endocrine pancreas was examined. The adult human duct cells expressed Pdx1/Ipf1 {Heimberg, et al. Diabetes 49: 571-579 (2000)}, but not ngn3, NeuroD/β2, Pax4, Pax6, Nkx2.2 or Nkx6.1. The expression of these transcription factors was regulated at the transcriptional level (FIG. 1A), except for Nkx6.1, which is subject to a post-transcriptional control {Watada et al. J. Biol. Chem. 275: 34224-34230 (2000)} and was undetectable in adult duct cells at the protein level (FIG. 1B). In contrast with mouse islets {Smith et al. Mol. Cell. Biol. 19: 8272-8280 (1999)} or rat purified β-cells (our own results), Pax4 was consistently expressed in mature human endocrine pancreas (FIG. 1A). Compared to adult human islet cells, the level of Notch 1, -2 and -3 transcripts (FIG. 3C), which encode the receptors that signal for lateral inhibition of endocrine cell differentiation {Jensen et al. Nat. Genet. 24:36-44 (2000)} {Lammertet al. Mech. Dev. 94: 199-203 (2000)} was high in duct cells, the expression level of Notch ligands Jagged1 and -2 was similar and that ofDll1, -3 and -4 was low (FIG. 3C). Synaptophysin, chromogranin A, prohormone convertase 1/3 and -2, insulin, glucagon, glucose transporter type II and glucokinase, all proteins known to support the mature endocrine phenotype were present in human islets but absent from duct cells (FIG. 5A and not shown).

Ectopic Expression of Neurogenin 3 Activates Key Endocrine Transcription Factor Genes.

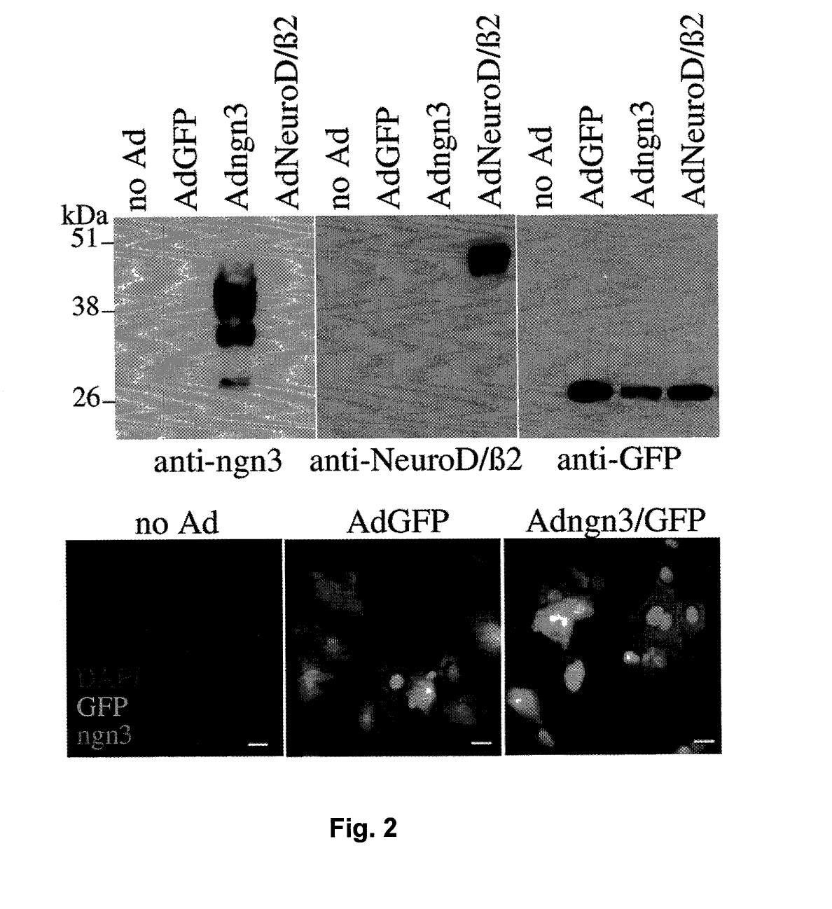

Ngn3 and its downstream target NeuroD/β2 were ectopically expressed in duct cells using the recombinant adenovirusses (rAds) {He, et al. Proc.Natl. Acad. Sci. USA 95: 2509-2514 (1998)} Adngn3 and AdNeuroD/β2 that carry the cognate transgenes ngn3 and NeuroD/β2 respectively under the control of a constitutive CMV-promotor (FIG. 2). Both the rAds used in this study co-expressed green fluorescent protein (GFP) as a reporter and parallel infections with AdGFP served as a control for non-specific viral effects. A multiplicity of infection of 50 was used because of a favorable balance between infection efficiency (30 to 40% GFP expression, FIG. 2) and cell survival (more than 85% living cells) and whether the infected duct cells were cultured as aggregates in suspension or as monolayers, did not affect the consequences of the infection.

The results obtained from the infected cells demonstrated that Adngn3 and AdNeuroD/β2 both stimulated the expression of NeuroD/β2 (FIG. 3A) and binding of ngn3 to the NeuroD/β2 promotor {Huang, et al. Mol. Cell. Biol. 20:3292-3307 (2000)} was confirmed by gel retardation ofE box-1 and E box-3 sequences following binding of nuclear proteins extracted from Adngn3-infected duct cells (data not shown). While the genes encoding Pdx1/Ipf1, Nkx6.1 or Pax6 were not upregulated or activated by overexpression of ngn3 or NeuroD/β2 (data not shown), the expression of Pax4 and Nkx2.2, both known to be essential for embryonic β-cell formation {Sosa-Pineda, et al. Nature 386: 399-402 (1997)} {Sussel, et al. Development 125: 2213-2221 (1998)} was clearly induced from 3 days post-infection on (FIG. 3A. Compared to NeuroD/β2, Pax4 and Nkx2.2, the abundance of Nkx6. 1-encoding mRNA was only slightly affected by ectopic expression of either ngn3 or NeuroD/β2. The -upregulation ofNeuroD/β2, Pax4 and Nkx2.2 was confirmed by in situ hybridization or immunocytochemistry (FIG. 3B). The Nkx6.1 protein, which is fundamental for embryonic β-cell development (Sander et al., 2000 Development 127: 3533-3542), appeared in duct cells infected with Adngn3 and AdNeuroD/β2. Ten days following infection, many cells expressed either Nkx2.2 or ngn3 (FIG. 3B, panel f., arrowheads), rarely both. Some Nkx2.2-expressing cells, however, still contained GFP (FIG. 3B, panel f., arrow). In addition, Dll1 and Dll4, but not Dll3, were activated by ectopic expression of ngn3 (FIG. 3C). Although the duct cell preparation contained less than 1% insulin-positive cells, their influence on the ngn3 or NeuroD/β2 effects can not be ruled out. However, infection of PC12, a clonal cell line of neuroendocrine origin that completely lacks insulin gene expression, with Adngn3 or with AdNeuroD/β2 showed that both ngn3 and NeuroD/β2 activated Pax4 (but not Nkx6.1) and stimulated the endogenous NeuroD/β2 and Nkx2.2 gene expression (FIG. 4A). In contrast, Adngn3 or AdNeuroD/β2 failed to induce Pax4, NeuroD/β2 or Nkx2.2 in HeLa cells, a non-neuroendocrine human cell line (data not shown). These data demonstrate that the effects of ngn-3 and NeuroD/β2 are autonomous and cell type specific.

Ectopic Expression of Neurogenin 3 and AdNeuroD/β2 in Adult Human Duct Cells Induces Expression of Endocrine Markers Including Insulin.

Expression of a number of endocrine markers was also examined in Adngn3 and AdNeuroD/β2 infected cells. By day 10 following infection of adult human duct cells with either Adngn3 or AdNeuroD/β2 gene expression of synaptophysin, chromogranin A, prohormone convertase PC1/3, glucokinase and insulin, all proteins that characterize a differentiated endocrine phenotype, was activated (FIG. 5A). By comparison, neither Ngn-3 nor NeuroD/β2 had any effect on the abundance of Glut-2, glucagon or somatostatin transcripts (data not shown).

The number of synaptophysin- and insulin-positive duct cells, as well as the intracellular insulin content and the amount of insulin that accumulated in the medium also significantly increased following infection with Adngn3 or AdNeuroD/β2 compared to AdGFP-infected cells (Table and FIG. 5B).

| | insulin | insulin | | |
|---|---|---|---|---|
| | content | release | insulin⁺ | synaptophysin⁺ |
| | % versus | % versus | % of total | % of total |
| | AdGFP | AdGFP | cells | cells |
| | n = 4 | n = 3 | n = 4 | n = 5 |
| AdGFP | 100 ± 0 | 100 ± 0 | 0.8 ± 0.1 | 1.4 ± 0.3 |
| AdNgn-3 | 250 ± 60* | 340 ± 20* | 13.0 ± 3.0* | 21.5 ± 3.6* |
| AdNeuroD/β2 | 310 ± 50* | 330 ± 20* | 7.5 ± 1.7* | 9.5 ± 1.1* |

Table I:

Effect of Ngn-3 or NeuroD/β2 on relative concentration of insulin in duct cells and -medium (accumulated during 48H) and on the percentage of insulin- and synaptophysin-immunostained cells. *:p<0.05 versus AdGFP-infected duct cells as determined by the paired Student's t-test.

At day ten, the fraction of cells that expressed the endocrine markers was high and generally differed from the ngn3-expressing cells (FIG. 5B, panels b., c. and h.: arrowheads point to single-positive cells), whose number was decreased compared to earlier time points following infection. The rare cells that co-expressed endocrine markers and ngn3 contained only low levels ngn3 as compared to the single-positive cells (FIG. 5B, b. and h.: arrows). More than 90% of the synaptophysin-expressing cells are CK19-positive duct cells (FIG. 5B, panel i.) but only few insulin-positive cells (still) contained the duct cell marker (FIG. 5B, panel j.).

Effects on the (neuro)endocrine-specific gene expression are difficult to study in the PC 12 cell line as it contains high endogenous levels of neuroendocrine transcripts. Nonetheless, analysis of endocrine-specific transcripts revealed that both ngn3 and NeuroD/β2 induced expression of the insulin gene (FIG. 4A) and the appearance of insulin following ngn3 overexpression was confirmed by immunostaining (FIG. 4B). By comparison, neither Ngn-3 nor NeuroD/β2 affected expression of the genes encoding glucokinase, Glut2, somatostatin or glucagon (data not shown).

Materials And Methods for Example 5B

Production of Recombinant Adenovirusses.

Adenoviral vectors were made available by Drs. T-C. He and B. Vogelstein (Johns Hopkins Oncology Center, Baltimore, Md.). Recombinant, replication-deficient adenovirusses expressing Green Fluorescent Protein (AdGFP), mouse ngn3, in combination with GFP (Adngn3) or rat NeuroD/β2, in combination with GFP (AdNeuroD/β2) were constructed following the standard protocol {He, Zhou, et al. Proc. Natl. Acad. Sci. USA 95: 2509-2514 (1998)}.

Cell Isolation and Cell Culture.

Adult human pancreata from heart-beating cadaveric nondiabetic donors were procured by European hospitals affiliated with the Eurotransplant Foundation (Leiden, The Netherlands) and processed by P-Cell Transplant, a multicenter program involving β-cell transplantation in type 1 diabetic patients. The exocrine cell preparation was cultured in suspension in Ham's F10 (Bio-Whittaker, Verviers, Belgium), 0.5% Bovine Serum Albumin (BSA) (Boehringer Mannheim, Mannheim, Germany), 7.5 mM glucose, 100 U/ml penicillin, 100 μg/ml streptomycin and 1 mM L-glutamine at 37° C. in a humidified atmosphere of 5% CO₂. 24 hours after isolation, the cells were washed and the medium was renewed. On day four of culture, cells were counted and were either infected and further cultured in suspension. Alternatively, day four cells were plated to form monolayers in the presence of 5% fetal bovine serum (FBS) (Life Technologies, Rockville, Md.) and infected 6 days later. Under all conditions, cell culture medium was renewed every other day.

PC12 cells were cultured in suspsension in RPMI 1640 with Glutamax (Life Technologies, Rockville, Md.), 10% FBS, 100 U/ml penicillin, 100 μg/ml streptomycin at 37° C. in a humidified atmosphere of 5% CO₂.

Viral Infection of Isolated Exocrine Cells and PC12 Cells.

Cells were either infected with control virus (AdGFP) or with Adngn3 or AdNeuroD/β2 as described previously {Heimberg, et al. Diabetes 50: 2219-2224 (2001)}. Adult human duct cells were infected at a multiplicity of infection (MOI) of 50, for 4 hours at 37° C. PC12 cells were infected at MOI 20 for 4 hours at 37° C.

Protein Analysis

Immunohistochemical analysis was performed on 4 μm thick paraffin sections by indirect immunofluorescence as described {Heimberg, et al. Diabetes 49: 571-579 (2000)}. Similar methods were applied to cells from suspension cultures after fixation for 1 hour in 4% paraformaldehyde and pelleting in 2% agarose before paraffin embedding. Virus-infected cells were harvested 3 days postinfection for analysis of transcription factor and Dll/Notch expression (FIGS. 2, 3B, and 4B) and virus-infected cells were harvested 10 days postinfection for analysis of endocrine marker expression (FIG. 5B). Before incubation with the first antibody, sections for CK19 staining were trypsin-treated.

PC1/3 antiserum was from Dr. I. Lindberg (Louisiana State University Health Sciences Center, New Orleans, La.) {Vindrola & Lindberg Mol. Endocrinol. 6: 1088-1094 (1992)}, anti-ngn3 from Dr. M. German (University of California San Francisco, San Francisco, Calif.) and anti-insulin from Dr. C. Van Schravendijck (Free University of Brussels, Brussels, B). The anti-Nkx2.2 was developed by Dr. T. Jessell and obtained from the DSHB (University of Iowa, Iowa City, Iowa). Anti-Nkx6.1 has been described before {Oster et al (1998) Cytochem. 46: 717-721}. Anti-chromogranin A was from Biogenex (San Ramon, Calif.) anti-synaptophysin from Novocastra (Newcastle upon Tyne, UK), anti-cytokeratin 19 from Dako (Santa Barbara, Calif.) and anti-HA from Boehringer Mannheim (Mannheim, Germany). Secondary antibodies were Cy3- or FITC.-labelled anti-rabbit, anti-mouse and anti-guinea pig (Jackson ImmunoResearch). Immunoblot analysis was done as described before {Heimberg, et al. Diabetes 49: 571-579 (2000)}.

mRNA analysis.

Total RNA was isolated using RNEasy columns (Qiagen, Valencia, Calif.) and reverse transcription followed by the polymerase chain reaction were performed and analyzed as described {Heimberg, et al. Proc. Natl. Acad. Sci. USA 93:7036-7041 (1996)}. For RNA analysis of virus-infected cells for transcription factor and Dll/Notch expression (FIGS. 3A, 3C and 4A), the virus-infected cells were harvested at 3 days postinfection. For RNA analysis of virus-infected cells for endocrine marker expression (FIG. 5A), the virus-infected cells were harvested at 10 days postinfection. Primers were:

| | |
|---|---|
| human Pdx 1/Ipf1 (277 bp): | |
| 5′-CTGCCTTTCCCATGGATGAA (forward), | (SEQ ID NO:23) |
| 5′-CGCTTCTTGTCCTCCTCCTTT (re- | (SEQ ID NO:24) |
| verse), | |
| human ngn3 (286 bp): | |
| 5′-AGACGACGCGAAGCTCACC (forward), | (SEQ ID NO:25) |
| 5′-AAGCAGACTGCCTGGGCT (reverse), | (SEQ ID NO:26) |
| human NeuroD/β2(439 bp): | |
| 5′-ATCCCAACCCACCACCAACC (forward), | (SEQ ID NO:27) |
| 5′-CAGCGGTGCCTGAGAAGATT (reverse), | (SEQ ID NO:28) |
| human Pax4 (496 bp): | |
| 5′-AGGAGGACCAGGGACTACCGT (for- | (SEQ ID NO:29) |
| ward), | |
| 5′-TTTAGGTGGGGTGTCACTCAG (re- | (SEQ ID NO:30) |
| verse), | |
| human Pax6 (301 bp): | |
| 5′-CAAAAGTCCAAGTGCTGGACAA (for- | (SEQ ID NO:31) |
| ward), | |
| 5′-CCCATCTGTTGCYFTTCGCT (reverse), | (SEQ ID NO:32) |
| human Nkx2.2 (329 bp): | |
| 5′-TGCAGCACATGCAGTACAACG (for- | (SEQ ID NO:33) |
| ward), | |
| 5′-TCCCAAGGTTCAGAAGGAGAGG (re- | (SEQ ID NO:34) |
| verse), | |
| human Nkx6.1 (284 bp): | |
| 5′-TCTTCTGGCCCGGGGTGATG (forward), | (SEQ ID NO:35) |
| 5′-AGCCGCGTGCTTCTTCCTCC (reverse), | (SEQ ID NO:36) |
| human synaptophysin (214 bp) | |
| 5′-GCCACATGCGGCAGCTACAG (forward), | (SEQ ID NO:37) |
| 5′-ACACGGCCACGGTGACAAAG (reverse), | (SEQ ID NO:38) |
| human chromogranin A (286 bp): | |
| 5′-CCGCTGTCCTGGCTCTTCT (forward), | (SEQ ID NO:39) |
| 5′-CCGCTGTGTTTCTTCTGCTG (reverse), | (SEQ ID NO:40) |
| human/rat insulin (438 bp): | |
| 5′-GCAGCCTTTGTGGAACCAACA (for- | (SEQ ID NO:41) |
| ward), | |
| 5′-TCTGCGGTCATCAAATGAGG (reverse), | (SEQ ID NO:42) |
| human glucagon (221 bp): | |
| 5′-CCCAAGATTTTGTGCAGTGGTT (for- | (SEQ ID NO:43) |
| ward), | |
| 5′-GCGGCCAAGTTCTTCAACAAT (re- | (SEQ ID NO:44) |
| verse), | |
| human glucokinase: | |
| 5′-CTGGATGACAGAGCCAGGAT (forward), | (SEQ ID NO:45) |
| 5′-TCACCATTGCCACCACATCCAT (re- | (SEQ ID NO:46) |
| verse), | |
| human PC 1/3 (355 bp): | |
| 5′-CAAGATACCAGGATGACGGCA (for- | (SEQ ID NO:47) |
| ward), | |
| 5′-GCCTCAATAGCATCCGTCACA (re- | (SEQ ID NO:48) |
| verse), | |
| mouse ngn3 (288 bp) | |
| 5′-CCGGATGACGCCAAACTTACA (for- | (SEQ ID NO:49) |
| ward), | |
| 5′-ACACCAGTGCTCCCGGGAG (reverse), | (SEQ ID NO:50) |
| rat NeuroD/β2 (300 bp): | |
| 5′-GGACTTTCTTGCCTGAGCAGA (for- | (SEQ ID NO:51) |
| ward), | |
| 5′-AACTCGGTGGATGGTTCGTGT (re- | (SEQ ID NO:52) |
| verse), | |
| rat Pax4 (224 bp): | |
| 5′-ATGCGACCCTGTGACATCTCA (for- | (SEQ ID NO:53) |
| ward), | |
| 5′-AAGCCCTTCAGCACAAAGCTG (re- | (SEQ ID NO:54) |
| verse), | |
| rat Nkx2.2 (209 bp): | |
| 5′-CATGTCGCTGACCAACACAAAG (for- | (SEQ ID NO:55) |
| ward), | |
| 5′-TCGCTGCTGTCGTAGAAAGGA (re- | (SEQ ID NO:56) |
| verse), | |
| rat/human β-actin: | |
| 5′-AGAGCTATGAGCTGCCTGAC (forward), | (SEQ ID NO:57) |
| 5′-CTGATCCACATCTGCTGGAA (reverse), | (SEQ ID NO:58)) |
| human Delta4 (forward): | |
| 5′-ACCACTTCCGGCCACTATGTGT, | (SEQ ID NO:59) |
| 5′- TCTTGGTCACAAAACAGGCCT (re- | (SEQ ID NO:60) |
| verse), | |
| human Delta3 (forward): | |
| 5′-TCAACAACCTAAGGACGCAGG, | (SEQ ID NO:61) |
| 5′- TTCACGGACAGAATCGAGGAAG (re- | (SEQ ID NO:62) |
| verse), | |
| human Deltal (forward) | |
| 5′- CCTGATGACCTCGCAACAGAA, | (SEQ ID NO:63) |
| 5′-CATGCTGCTCATCACATCCAG (re- | (SEQ ID NO:64) |
| verse), | |
| human Notchi (forward): | |
| 5′-GAATCCAACCCTTGTGTCAAC, | (SEQ ID NO:65) |
| 5′-GCAACGTCGTCAATACACGTG (reverse) | (SEQ ID NO:66) |
| human Notch2 (forward): | |
| 5′-CGCTGCATTGACCTGGTCAAT, | (SEQ ID NO:67) |
| 5′-TACATGTTGCACCCTTGCGA (reverse), | (SEQ ID NO:68) |
| human Notch3 (forward) | |
| 5′-GGCATTGCTAGCTTCTCGTGT, | (SEQ ID NO:69) |
| 5′-CATAACGGTTGATGCCATCAC(G) (re- | (SEQ ID NO :70) |
| verse). | |

For in situ hybridisation, human-specific NeuroD/β2, Pax4 and ngn3 PCR-products were subcloned into the pGEM-T Easy vector (Promega, Madison, Wis.) and Sal I-linearized (Life Technologies). Digoxigenin-labeled transcripts were produced according to manufacturer's instructions (MAXI Script, Ambion, Austin, Tex.). RNA in situ hybridisation was performed as described {Petersenet al Mol. Cell. Biol. Res. Comm. 3: 249-254 (2000)} {Gradwohl, et al. Proc. Natl. Acad. Sci. USA 97:1607-1611 (2000)}. Detection of hybridized probes made use of an alkaline phosphatase-labeled anti-digoxigenin antibody (Boehringer Mannheim) and the substrate BM-Purple (Boehringer Mannheim).

Data Analysis.

Results obtained from infected cells were compared to uninfected and/or control virus-infected cells, and statistically analyzed using the paired Student's T-test.

### Example 6

### TGF-β Superfamily in Endocrine Development

The objective is to determine the identity, expression pattern, and function of the TGF-βs, TGF-β-R's, and TGF-β antagonists that are expressed during pancreas development. As described above, experiments are conducted using RT-PCR screening of embryonic pancreas RNA using primers for all known TGF-βs, TG-β-R's, and antagonists followed by in situ hybridization to analyze cellular expression. As described above, functional studies utilize gain- and loss-of-function experiments in ex plant cultures of rat and mouse embryonic pancreas, knockout mice, and chicken embryos, followed by analysis of pancreas development using relevant markers.

Results:

RT-PCR screening embryonic pancreas RNA identified ligands TGF-β1, -2, -3, BMP2, -3B, -4, -5, -6, -7, -11, inhibin-αa, inhibin-βA, βB, βC, βE; receptors ALK2, -3, -4, 6, -7, BMPRII, ActRIIA, ActRIIB, and TβRII. Antagonists identified included Follistatin, FREL, FLIK, Cerberus, DAN, PRDC, Chordin, Noggin, and Gremlin. In situ hybridization showed that follistatin appear at day e14, and that FREL is expressed throughout embryonic development.

### Example 7

### Notch Signaling in Endocrine Development

Experiments are conducted to determine the identity, expression pattern, and function of the Notch receptors, ligands, and Notch modifying glycosyltransferases expressed during pancreas development. As described above, RT-PCR screening of embryonic pancreas RNA is conducted using primers for all known Notch receptors, ligands, and Notch modifying glycosyltransferases, followed by in situ hybridization to analyze cellular expression. Functional studies utilize gain- and loss-of-function experiments in ex plant cultures of rat and mouse embryonic pancreas, knockout mice, and chicken embryos, followed by analysis of pancreas development using relevant markers.

Results:

In situ hybridization revealed expression of the receptors Notch1 and -2 in embryonic epithelium and Notch3 in the mesenchyme. The ligands Dll1 and Jagged-1 were found to be expressed in the epithelium, and Jagged-2 in endothelial cells. Loss-of function of HES1, Dll-1 and the transcription factors RBP-J all display excessive endocrine development. Gain-of-function experiments with constitutive active Notch1 expressed in embryonic epithelium blocked acinar differentiation and branching morphogenesis and significantly reduce the number of endocrine cells developing.

### Example 8

### Insulin-IGF-NGF Signaling in β-Cell Survival

Experiments are conducted to determine whether and to what extent insulin/IGFs and NGF-family growth factors can protect β-cells against cytokine mediated β-cell apoptosis. Ultimately, the experiments aim to identify signaling pathways required for the protective effects.

Isolated islets are subjected to cytokine treatment, and the effect of insulin/IGFs and NGF-family growth factors tested for their efficacy in protection against apoptosis. The signaling pathways involved are determined using adenoviral gene transfer of relevant dominant negative and constitutive active kinases into islets followed by the growth factor protection assays.

### Example 9

### GLP-1 Induction of β-Cell Regeneration

In vitro studies were conducted investigating the signaling pathway for GLP-1 induced β-cell proliferation. Results of the in vitro experiments demonstrated GLP-1 and exendin-4 stimulate proliferation of cultured β-cells and lead to increased β-cell volume.

The studies were repeated in vivo with ZDF rats and db/db mice. The studies with ZDF rats confirmed that GLP-1 is able to increase β-cell volume. In db/db mice, a significant stimulation of both proliferation and increased β-cell volume was seen with GLP-1. In 60% pancreatectomized (PX) rats, a small significant lowering of blood glucose measured in an OGTT following treatment with GLP-1 relative to sham treated animals. Preliminary histological results showed that GLP-1 treatment resulted in larger islets and more insulin compared to sham treated animals. These results indicate that GLP-1 compounds have a proliferation/mitogenic β-cell potential as part of their mechanism of action.

### Example 10

### Transplantation of in vitro Generated Mature Pancreatic β-Cells

The in vitro generated mature pancreatic β-cell produced by the protocol described in Example 5, by virtue of its retention of normal beta cell characteristics with regard to insulin secretion and blood glucose regulation, offers an alternative to insulin administration in the treatment of diabetes in both animals and humans. Accordingly, the present invention also provides a method for treating a diabetic subject. The method comprises implanting the insulin-secreting cells produced by the method of the invention in the body of the diabetic subject in an amount effective to establish and maintain physiologically acceptable levels of blood glucose. The cells may be implanted in any feasible location within the body where they come in contact with the blood stream of the recipient. Suitable locations include but are not limited to the peritoneal cavity and the pancreas. Other locations would be apparent to one skilled in the art. The mature pancreatic islet cells may be implanted by methods known to those skilled in the art such as by surgical means, injection and the like. In one embodiment, the effective amount of pancreatic islet cells is preferably about 100 to about 300 million cells. The effective amount of pancreatic islet cells, however, will depend upon the method of implantation, the pharmacokinetic characteristics of the subject treated, and/or the presence of other diseases or conditions. Such amounts are readily determined by one skilled in the art.

Cell Implantation.

Islet cells generated as described in Example 5 are trypsinized, washed in PBS, and resuspended in PBS at 5×10<6>cells/ml. Recipient mice, for example, C3H mice, are injected intraperitoneally (I.P.) with 106 cells each and maintained on regular drinking water or on water containing 1 mg/ml Tc and 2.5% sucrose. The transplanted mice are monitored weekly for blood glucose using Glucometer strips. To generate diabetic mice, 12 mice are injected I.P. with one dose of 200 mg streptozotocin (Sigma) per kg of body weight, followed 7 days later by 3 doses of 50 mg/kg on 3 consecutive days, which causes hyperglycemia within 6-9 additional days. Mice are monitored weekly for blood glucose levels. Euglycemia is obtained in the cell-implanted group relative to the control mice.

1. A method for generating insulin-secreting cells from precursor stem cells, said method comprising exposing said precursor cells to a nucleic acid molecule encoding neurogenin3 (ngn3) under conditions effective to generate said insulin-secreting cells from said precursor cells.

2. The method of claim 1, wherein the precursor cells are embryonic stem cells.

3. The method of claim 1, wherein said precursor cells are exposed in vitro to said nucleic acid molecule encoding ngn3.

4. A method for generating insulin-secreting cells from precursor stem cells, said method comprising exposing said precursor cells to an activator of ngn3 gene expression under conditions effective to generate said insulin-secreting cells from said precursor cells.

5. The method of claim 4, wherein said precursor cells are embryonic stem cells.

6. The method of claim 4, wherein said precursor cells are exposed in vitro to said activator of ngn3 gene expression.

7. A method for generating insulin-secreting cells from precursor stem cells, said method comprising exposing said precursor cells to a nucleic acid molecule encoding NeuroD/β2 under conditions effective to generate said insulin-secreting cells from said precursor cells.

8. The method of claim 7, wherein said precursor cells are embryonic stem cells.

9. The method of claim 7, wherein said precursor cells are exposed in vitro to said nucleic acid molecule encoding NeuroD/β2.

10. A method for generating insulin-secreting cells from precursor stem cells, said method comprising exposing said precursor cells to an activator of NeuroD/β2 gene expression under conditions effective to generate said insulin-secreting cells from said precursor cells.

11. The method of claim 10, wherein said precursor cells are embryonic stem cells.

12. The method of claim 10, wherein said precursor cells are exposed in vitro to said activator of NeuroD/β2 gene expression.

13. A method for generating insulin-secreting cells from adult pancreatic exocrine cells, said method comprising exposing said exocrine cells to a nucleic acid molecule encoding ngn3 under conditions effective to generate said insulin-secreting cells from said exocrine cells.

14. The method of claim 13, wherein said exocrine cells are pancreatic duct cells.

15. The method of claim 14, wherein said duct cells are human cells.

16. The method of claim 13, wherein said exocrine cells are exposed in vitro to said nucleic acid molecule encoding ngn3.

17. A method for generating insulin-secreting cells from adult pancreatic exocrine cells, said method comprising exposing said exocrine cells to an activator of ngn3 gene expression under conditions effective to generate said insulin-secreting cells from said exocrine cells.

18. The method of claim 17, wherein said exocrine cells are pancreatic duct cells.

19. The method of claim 18, wherein said duct cells are human cells.

20. The method of claim 17, wherein said exocrine cells are exposed in vitro to said activator of ngn3 gene expression.

21. A method for generating insulin-secreting cells from adult pancreatic exocrine cells, said method comprising exposing said exocrine cells to a nucleic acid molecule encoding NeuroD/β2 under conditions effective to generate said insulin-secreting cells from said exocrine cells

22. The method of claim 21, wherein said exocrine cells are pancreatic duct cells.

23. The method of claim 22, wherein said duct cells are human cells.

24. The method of claim 21, wherein said exocrine cells are exposed in vitro to said nucleic acid molecule encoding NeuroD/β2.

25. A method for generating insulin-secreting cells from adult pancreatic exocrine cells, said method comprising exposing said exocrine cells to an activator of NeuroD/β2 gene expression under conditions effective to generate said insulin-secreting cells from said exocrine cells.

26. The method of claim 25, wherein said exocrine cells are pancreatic duct cells.

27. The method of claim 26, wherein said duct cells are human cells.

28. The method of claim 25, wherein said exocrine cells are exposed in vitro to said activator of NeuroD/β2 gene expression.

29. An insulin-secreting cell produced by the method of claim 1.

30. An insulin-secreting cell produced by the method of claim 4.

31. An insulin-secreting cell produced by the method of claim 7.

32. An insulin-secreting cell produced by the method of claim 10.

33. An insulin-secreting cell produced by the method of claim 13.

34. An insulin-secreting cell produced by the method of claim 17.

35. An insulin-secreting cell produced by the method of claim 21.

36. An insulin-secreting cell produced by the method of claim 25.

37. A method for identifying whether a compound is an activator of ngn3 gene expression, said method comprising: (a) exposing progenitor stem cells or adult pancreatic exocrine cells to said compound in vitro and (b) measuring the generation of insulin-secreting cells from said exposed progenitor cells or adult pancreatic exocrine cells, where the generation of insulin-secreting cells from said exposed progenitor cells or adult pancreatic exocrine cells indicates that said compound is an activator of ngn3 gene expression.

38. A method for identifying whether a compound is an activator of NeuroD/β2 gene expression, said method comprising: (a) exposing progenitor stem cells or adult pancreatic exocrine cells to said compound in vitro and (b) measuring the generation of insulin-secreting cells from said exposed progenitor cells or adult pancreatic exocrine cells, where the generation of insulin-secreting cells from said exposed progenitor cells or adult pancreatic exocrine cells indicates that said compound is an activator of NeuroD/β2 gene expression.

39. A method for generating insulin-secreting cells from precursor stem cells or adult pancreatic exocrine cells, said method comprising exposing said cells to a compound identified by the method of claim 37 in an amount effective to generate said insulin-secreting cells.

40. A method for generating insulin-secreting cells from precursor stem cells or adult pancreatic exocrine cells, said method comprising exposing said cells to a compound identified by the method of claim 38 in an amount effective to generate said insulin-secreting cells.

41. An isolated insulin-secreting cell, wherein said cell is characterized by the absence of RNA transcripts for glucose transporter type 2 protein and by the presence of RNA transcripts for synaptophasin, chromogranin A, prohormone convertase PC1/3, and glucokinase.
